(19) United States
(12) Patent Application Publication
(10) Pub. No.: US 2025/0066423 A1
(43) Pub. Date: Feb. 27, 2025

(54) SESTRIN-MAPK COMPLEX INHIBITORS

(71) Applicant: Sentcell Ltd, London (GB)

(72) Inventor: Alessio Lanna, Colleferro, Roma (IT)

(73) Assignee: Sentcell Ltd, London (GB)

(21) Appl. No.: 18/722,786

(22) PCT Filed: Dec. 24, 2021
(86) PCT No.: PCT/IT2021/000059

§ 371 Date: Jun. 21, 2024

(51) Int. Cl. C07K 7/64; A61K 38/00

(52) U.S. Cl. CPC C07K 7/64; A61K 38/00

(57) ABSTRACT

The invention provides a cyclic polypeptide, derivative or analogue thereof, comprising or consisting of an amino acid sequence derived from sestrin, or a truncation thereof. The cyclic polypeptides can be used in treating, preventing or ameliorating infectious diseases (viral and bacterial), and age-related conditions, such as cancer and neurodegenerative disorders, and particularly conditions that are associated with or caused by impairment of the immune system response, which is mediated by T cells.

The present invention relates to inhibitors of sestrin-MAPK complexes (sMAC), herein also referred to as disruptors of sMAC (i.e., “DOS”), and particularly, although not exclusively, to cyclic polypeptide (DOS) inhibitors of sMAC. The invention also relates to the use of these DOS compounds in treating, preventing or ameliorating infectious diseases (viral, bacterial and protist), and age-related conditions, such as cancer and neurodegenerative disorders. The invention is especially concerned with treating conditions that are associated with or caused by aging, or impairment of the immune system response, which is mediated by T cells. The invention also extends to pharmaceutical compositions comprising the DOS compounds, and to methods of making such DOS compounds.

The worldwide demographic is shifting towards an older age, with the aged population expected to reach over two billion by 2050 (Lutz et al., 2008). One challenge posed to healthy aging is immune-senescence, which predisposes older individuals to an increased risk of cancer, neurodegenerative and infectious diseases, including influenza and other life-threatening outbreaks, such as the most recent coronavirus pandemic (Akbar et al., 2016; Dorshkind et al., 2009). While vaccination is an efficient way to build up immunity in younger people, the effectiveness of any vaccination strategy in the elderly and immunocompromised is severely impaired, causing increased hospitalisations with high morbidity and mortality rates (Goronzy and Weyand, 2013; Levin, 2012; Yousefzadeh et al., 2021). Currently, strategies to restore long-lasting immune protection in the elderly are lacking, especially approaches that aim to rejuvenate the immune system, and stop the spread of infections among vaccinated individuals. There is, therefore, an urgent need to implement new strategies to improve quality of life during aging.

Immune cells that exhibit features of senescence increase during aging (Weng et al., 2009). In fact, in older individuals, over 50% of the immune cells are senescent. As T cells progress towards senescence, there is a sequential loss of the costimulatory receptors CD27 and CD28. Early stage CD4+ T cells are CD27+ CD28+, those at an intermediate stage are CD27−CD28+, and the senescent CD4+ T cell population is CD27−CD28− (Lanna et al., 2014).

Stress-molecules, known as sestrins, are encoded in humans by the Sesn1, Sesn2 and Sesn3 genes. Sestrins accumulate in T cells, and bind to, and activate, the AMP-responsive protein kinase (AMPK) (Lanna et al., 2017). AMPK is a heterotrimeric protein consisting of the catalytic a subunit and the regulatory β and y subunits that are activated in response to increased intracellular AMP/ATP ratio. In turn, activation of AMPK coordinates global (ERK, JNK, p38) mitogen-activated protein kinase (MAPK) activation through auto-phosphorylation (see FIG. 1).

Three main subgroups of MAPKs have been identified, including extracellular regulated protein kinase (ERK), c-Jun N-terminal kinase (JNK) and p38 (Johnson and Lapadat, 2002). The MAPK signal transducing enzymes are involved in diverse aspects of mammalian physiology, including senescence, aging and metabolism. For example, as illustrated in FIG. 1, JNK inhibits TCR-CD28 signalling, p38 inhibits telomerase activity, and ERK increases DNA damage, all contributing to the accumulation of senescent T cells during aging (Lanna et al., 2017).

The inventors have previously shown that sestrin-MAPK complexes (sMAC), a large complex of immune-inhibitory proteins, accumulate in immunological cells (e.g. T cells) during aging, causing a senescent dysfunctional state (Lanna et al, Nature Immunology, 2014 and 2017; WO/2018/100410). Accordingly, the inventors have hypothesised that by targeting and inhibiting sestrins, sestrin-dependent sMAC activation can be disrupted, resulting in the prevention, delay or reversal of immune-senescence.

Currently, there are no known drugs that actively target and inhibit sMAC. Although certain experimental approaches, such as continuous administration of mTOR inhibitors, have been proposed to combat immune-senescence and improve vaccination efficacy in the elderly, these approaches have shown only modest effects (Mannick et al., 2014). For example, such approaches have not exceeded a 20% increase in antibody titres following vaccination (Mannick et al., 2014). Additionally, these approaches are associated with high costs and challenging compliance, since they must be administered for eight weeks, on a daily basis, before the vaccination protocol can begin (Mannick et al., 2014). Other experimental approaches with senolytics are far from ideal, because killing senescent immune cells to achieve rejuvenation may lead to severe immunodeficiency due to age-progressive thymic involution (Carpenter et al., 2021). Additionally, targeting sestrins by small molecule inhibitors to prevent sMAC activation is further complicated by the fact that sestrins lack enzymatic activities, and have no obvious catalytic domain to target (Budanov et al., 2010).

There is, therefore, a great need for improved therapies that can directly target and prevent sestrin-dependent sMAC activation and immune-senescence, and as a result, revive immunity during aging.

As described in the Examples, the inventors have generated novel inhibitors of sMAC, named DOS compounds (disruptors of sMAC). These DOS compounds are (in most embodiments) pentameric cyclic peptides, which inhibit sMAC in nanomolar amounts, by directly targeting sestrin for proteasomal degradation. Surprisingly, the inventors observed that these novel DOS compounds can provide long term immune protection with a single dose, both in the presence and absence of a vaccine, the latter being most unexpected. This is particularly advantageous when compared with mTORC1 inhibitors or AMPK activators that either require pre-administration (several weeks before immunization) with a vaccine (mTORC1 inhibitor) (Mannick et al., 2014) or need to be timed when the memory immune response arises (AMPK activator) (Pearce et al., 2009). The DOS compounds, although not inducing cell death, are instead able to reprogram the aged immune system, effectively ‘rejuvenating’ it. Advantageously, being able to rejuvenate, rather than kill, senescent cells, will preserve immunity in the elderly and potentially further extend human lifespan.

Thus, according to a first aspect of the invention, there is provided a cyclic polypeptide, derivative or analogue thereof, comprising or consisting of an amino acid sequence derived from sestrin, or a truncation thereof.

Sestrins are stress-inducible metabolic proteins, and humans express three sestrin proteins, sestrin 1, sestrin 2 and sestrin 3 (Lee et al., 2013). Each of the sestrin proteins are encoded by the genes Sesn1, Sesn2, and Sesn3, respectively.

Accordingly, in one embodiment, the cyclic polypeptide, derivative or analogue thereof, comprises or consists of an amino acid sequence derived from sestrin 1, or a truncation thereof.

The protein sequence of one isoform of human sestrin 1 (Gen Bank: 27244) is 492 amino acids in length, and is provided herein as SEQ ID No: 1, as follows:

[SEQ ID No: 1]
MRLAAAANEAYTAPLAVSGLLGCKQCGGGRDQDEELGIRIPRPLGQGPSR
FIPEKEILQVGSEDAQMHALFADSFAALGRLDNITLVMVFHPQYLESFLK
TQHYLLQMDGPLPLHYRHYIGIMAAARHQCSYLVNLHVNDFLHVGGDPKW
LNGLENAPQKLQNLGELNKVLAHRPWLITKEHIEGLLKAEEHSWSLAELV
HAVVLLTHYHSLASFTFGCGISPEIHCDGGHTFRPPSVSNYCICDITNGN
HSVDEMPVNSAENVSVSDSFFEVEALMEKMRQLQECRDEEEASQEEMASR
FEIEKRESMFVFSSDDEEVTPARAVSRHFEDTSYGYKDFSRHGMHVPTFR
VQDYCWEDHGYSLVNRLYPDVGQLIDEKFHIAYNLTYNTMAMHKDVDTSM
LRRAIWNYIHCMFGIRYDDYDYGEINQLLDRSFKVYIKTVVCTPEKVTKR
MYDSFWRQFKHSEKVHVNLLLIEARMQAELLYALRAITRYMT

The protein sequence of another isoform of human sestrin 1 (Gen Bank: 27244) is 551 amino acids in length, and is provided herein as SEQ ID No: 2, as follows:

[SEQ ID No: 2]
MAEGENEVRWDGLCSRDSTTRETALENIRQTILRKTEYLRSVKETPHRPS
DGLSNTESSDGLNKLLAHLLMLSKRCPFKDVREKSEFILKSIQELGIRIP
RPLGQGPSRFIPEKEILQVGSEDAQMHALFADSFAALGRLDNITLVMVFH
PQYLESFLKTQHYLLQMDGPLPLHYRHYIGIMAAARHQCSYLVNLHVNDF
LHVGGDPKWLNGLENAPQKLQNLGELNKVLAHRPWLITKEHIEGLLKAEE
HSWSLAELVHAVVLLTHYHSLASFTFGCGISPEIHCDGGHTFRPPSVSNY
CICDITNGNHSVDEMPVNSAENVSVSDSFFEVEALMEKMRQLQECRDEEE
ASQEEMASRFEIEKRESMFVFSSDDEEVTPARAVSRHFEDTSYGYKDFSR
HGMHVPTFRVQDYCWEDHGYSLVNRLYPDVGQLIDEKFHIAYNLTYNTMA
MHKDVDTSMLRRAIWNYIHCMFGIRYDDYDYGEINQLLDRSFKVYIKTVV
CTPEKVTKRMYDSFWRQFKHSEKVHVNLLLIEARMQAELLYALRAITRYM
T

The protein sequence of another isoform of human sestrin 1 (Gen Bank: 27244) is 426 amino acids in length, and is provided herein as SEQ ID No: 3, as follows:

[SEQ ID No: 3]
MHALFADSFAALGRLDNITLVMVFHPQYLESFLKTQHYLLQMDGPLPLHY
RHYIGIMAAARHQCSYLVNLHVNDFLHVGGDPKWLNGLENAPQKLQNLGE
LNKVLAHRPWLITKEHIEGLLKAEEHSWSLAELVHAVVLLTHYHSLASFT
FGCGISPEIHCDGGHTFRPPSVSNYCICDITNGNHSVDEMPVNSAENVSV
SDSFFEVEALMEKMRQLQECRDEEEASQEEMASRFEIEKRESMFVFSSDD
EEVTPARAVSRHFEDTSYGYKDFSRHGMHVPTFRVQDYCWEDHGYSLVNR
LYPDVGQLIDEKFHIAYNLTYNTMAMHKDVDTSMLRRAIWNYIHCMFGIR
YDDYDYGEINQLLDRSFKVYIKTVVCTPEKVTKRMYDSFWRQFKHSEKVH
VNLLLIEARMQAELLYALRAITRYMT

Thus, preferably the cyclic polypeptide, derivative or analogue thereof, comprises or consists of an amino acid sequence derived from sestrin 1, or a truncation thereof, wherein the sestrin 1 has an amino acid sequence substantially as set out in SEQ ID No: 1, 2 or 3, or a variant or fragment thereof.

Alternatively, in another embodiment, the cyclic polypeptide, derivative or analogue thereof, comprises or consists of an amino acid sequence derived from sestrin 2, or a truncation thereof.

The protein sequence of one isoform of sestrin 2 (Gen Bank: 83667) is 480 amino acids in length, and is provided herein as SEQ ID No: 4, as follows:

[SEQ ID No: 4]
MIVADSECRAELKDYLRFAPGGVGDSGPGEEQRESRARRGPRGPSAFIPV
EEVLREGAESLEQHLGLEALMSSGRVDNLAVVMGLHPDYFTSFWRLHYLL
LHTDGPLASSWRHYIAIMAAARHQCSYLVGSHMAEFLQTGGDPEWLLGLH
RAPEKLRKLSEINKLLAHRPWLITKEHIQALLKTGEHTWSLAELIQALVL
LTHCHSLSSFVFGCGILPEGDADGSPAPQAPTPPSEQSSPPSRDPLNNSG
GFESARDVEALMERMQQLQESLLRDEGTSQEEMESRFELEKSESLLVTPS
ADILEPSPHPDMLCFVEDPTFGYEDFTRRGAQAPPTFRAQDYTWEDHGYS
LIQRLYPEGGQLLDEKFQAAYSLTYNTIAMHSGVDTSVLRRAIWNYIHCV
FGIRYDDYDYGEVNQLLERNLKVYIKTVACYPEKTTRRMYNLFWRHFRHS
EKVHVNLLLLEARMQAALLYALRAITRYMT

Thus, preferably the cyclic polypeptide, derivative or analogue thereof, comprises or consists of an amino acid sequence derived from sestrin 2, or a truncation thereof, wherein the sestrin 2 has an amino acid sequence substantially as set out in SEQ ID No: 4, or a variant or fragment thereof.

Alternatively, in another embodiment, the cyclic polypeptide, derivative or analogue thereof comprises or consists of an amino acid sequence derived from sestrin 3, or a truncation thereof.

The protein sequence of one isoform of human sestrin 3 (Gen Bank: 143686) is 492 amino acids in length, and is provided herein as SEQ ID No: 5, as follows:

[SEQ ID No: 5]
MNRGGGSPSAAANYLLCTNCRKVLRKDKRIRVSQPLTRGPSAFIPEKEVV
QANTVDERTNFLVEEYSTSGRLDNITQVMSLHTQYLESFLRSQFYMLRMD
GPLPLPYRHYIAIMAAARHQCSYLINMHVDEFLKTGGIAEWLNGLEYVPQ
RLKNLNEINKLLAHRPWLITKEHIQKLVKTGENNWSLPELVHAVVLLAHY
HALASFVFGSGINPERDPEISNGFRLISVNNFCVCDLANDNNIENASLSG
SNFGIVDSLSELEALMERMKRLQEEREDEEASQEEMSTRFEKEKKESLFV
VSGDTFHSFPHSDFEDDMIITSDVSRYIEDPGFGYEDFARRGEEHLPTFR
AQDYTWENHGFSLVNRLYSDIGHLLDEKFRMVYNLTYNTMATHEDVDTTM
LRRALFNYVHCMFGIRYDDYDYGEVNQLLERSLKVYIKTVTCYPERTTKR
MYDSYWRQFKHSEKVHVNLLLMEARMQAELLYALRAITRHLT

The protein sequence of another embodiment of human sestrin 3 (Gen Bank: 143686) is 321 amino acids in length, and is provided herein as SEQ ID No: 6, as follows:

[SEQ ID No: 6]
MNRGGGSPSAAANYLLCTNCRKVLRKDKRIRVSQPLTRGPSAFIPEKEVV
QANTVDERTNFLVEEYSTSGRLDNITQVMSLHTQYLESFLRSQFYMLRMD
GPLPLPYRHYIAIMAAARHQCSYLINMHVDEFLKTGGIAEWLNGLEYVPQ
RLKNLNEINKLLAHRPWLITKEHIQKLVKTGENNWSLPELVHAVVLLAHY
HALASFVFGSGINPERDPEISNGFRLISVNNFCVCDLANDNNIENASLSG
SNFGIVDSLSELEALMERMKRLQEEREDEEASQEEMSTRFEKEKKESLFV
VSGDTFHSFPHSGAFLHFFAF

The protein sequence of another embodiment of human sestrin 3 (Gen Bank: 143686) is 353 amino acids in length, and is provided herein as SEQ ID No: 7, as follows:

[SEQ ID No: 7]
MSLHTQYLESFLRSQFYMLRMDGPLPLPYRHYIAIMKLVKTGENNWSLPE
LVHAVVLLAHYHALASFVFGSGINPERDPEISNGFRLISVNNFCVCDLAN
DNNIENASLSGSNFGIVDSLSELEALMERMKRLQEEREDEEASQEEMSTR
FEKEKKESLFVVSGDTFHSFPHSDFEDDMIITSDVSRYIEDPGFGYEDFA
RRGEEHLPTFRAQDYTWENHGFSLVNRLYSDIGHLLDEKFRMVYNLTYNT
MATHEDVDTTMLRRALFNYVHCMFGIRYDDYDYGEVNQLLERSLKVYIKT
VTCYPERTTKRMYDSYWRQFKHSEKVHVNLLLMEARMQAELLYALRAITR
HLT

Thus, preferably the cyclic polypeptide, derivative or analogue thereof, comprises or consists of an amino acid sequence derived from sestrin 3, or a truncation thereof, wherein the sestrin 3 has an amino acid sequence substantially as set out in SEQ ID No: 5, 6 or 7, or a variant or fragment thereof.

Preferably, however, the cyclic polypeptide, derivative or analogue thereof, comprises or consists of an amino acid sequence derived from sestrin 2, or a truncation thereof.

Cyclic polypeptides are peptide chains whose N- and C-termini are themselves linked together with a peptide bond that forms a circular chain of amino acids (Joo, 2012), and, to date, no cyclic peptides have been developed which comprise an amino acid sequence derived from any of sestrin 1, 2 or 3 (preferably sestrin 2), or a truncation thereof.

The term “derivative or analogue thereof” can mean a polypeptide within which amino acid residues are replaced by residues (whether natural amino acids, non-natural amino acids or amino acid mimics) with similar side chains or peptide backbone properties. Additionally, the terminals of such peptides may be protected by N- and/or C-terminal protecting groups with similar properties to acetyl or amide groups.

Derivatives and analogues of polypeptides according to the invention may also include those that increase the peptide's half-life in vivo. For example, a derivative or analogue of the peptides of the invention may include peptoid and retropeptoid derivatives of the peptides, peptide-peptoid hybrids and D-amino acid derivatives of the peptides.

Peptoids, or poly-N-substituted glycines, are a class of peptidomimetics whose side chains are appended to the nitrogen atom of the peptide backbone, rather than to the alpha-carbons, as they are in amino acids. Peptoid derivatives of the peptides of the invention may be readily designed from knowledge of the structure of the peptide. Retropeptoids (in which all amino acids are replaced by peptoid residues in reversed order) are also suitable derivatives in accordance with the invention. A retropeptoid is expected to bind in the opposite direction in the ligand-binding groove, as compared to a peptide or peptoid-peptide hybrid containing one peptoid residue. As a result, the side chains of the peptoid residues are able to point in the same direction as the side chains in the original peptide. D-amino acid derivatives of the peptides (rather than L-amino acids) decrease the unwanted breakdown of the peptide by proteases. The term “derived from” can mean an amino acid sequence which is a derivative or a modification of an amino acid sequence that is present in, or forms, sestrin 1, 2 or 3 (preferably sestrin 2), and portions thereof.

The term “truncation thereof” can mean the cyclic polypeptide derived from sestrin 1, 2, or 3 (preferably sestrin 2) is reduced in size by the removal of amino acids. The reduction of amino acids may be achieved by removal of residues from the C- and/or N-terminal of the peptide prior to cyclisation into the cyclic polypeptide of the invention, or may be achieved by deletion of one or more amino acids from within the core of the peptide prior to cyclisation.

Preferably, the cyclic polypeptide is purified and/or isolated, i.e. it is not found in nature, and even more preferably is de novo synthetized as later described.

It has been shown that the first 160 amino acid residues of sestrin 2 (SEQ ID No: 4) are believed to be involved in sestrin-AMPK interaction (Budanov and Karin, 2008). Accordingly, it is preferred that the cyclic polypeptide, derivative or analogue thereof comprises or consists of an amino acid sequence derived from the first 160 amino acids forming sestrin 2, or a truncation thereof, preferably wherein sestrin 2 comprises an amino acid sequence substantially as set out in SEQ ID No: 4, or a variant or fragment thereof.

Alternatively, in another embodiment, it is preferred that the cyclic polypeptide, derivative or analogue thereof comprises or consists of an amino acid sequence derived from the first 160 amino acids forming sestrin 1, or a truncation thereof, preferably wherein sestrin 1 comprises an amino acid sequence substantially as set out in SEQ ID No: 1, 2 or 3, or a variant or fragment thereof.

Alternatively, in another embodiment, it is preferred that the cyclic polypeptide, derivative or analogue thereof comprises or consists of an amino acid sequence derived from the first 160 amino acids forming sestrin 3, or a truncation thereof, preferably wherein sestrin 3 comprises an amino acid sequence substantially as set out in SEQ ID No: 5, 6 or 7, or a variant or fragment thereof. Most preferably, wherein sestrin 3 comprises an amino acid sequence substantially as set out in SEQ ID No: 5.

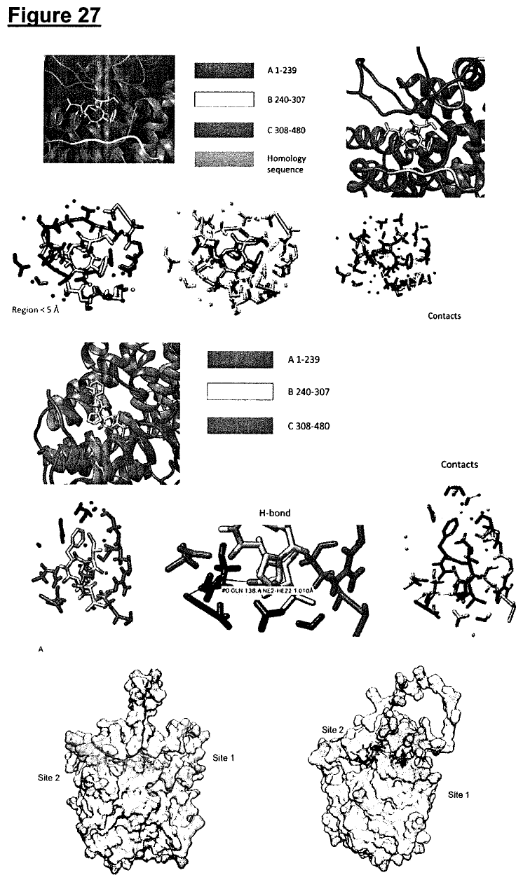

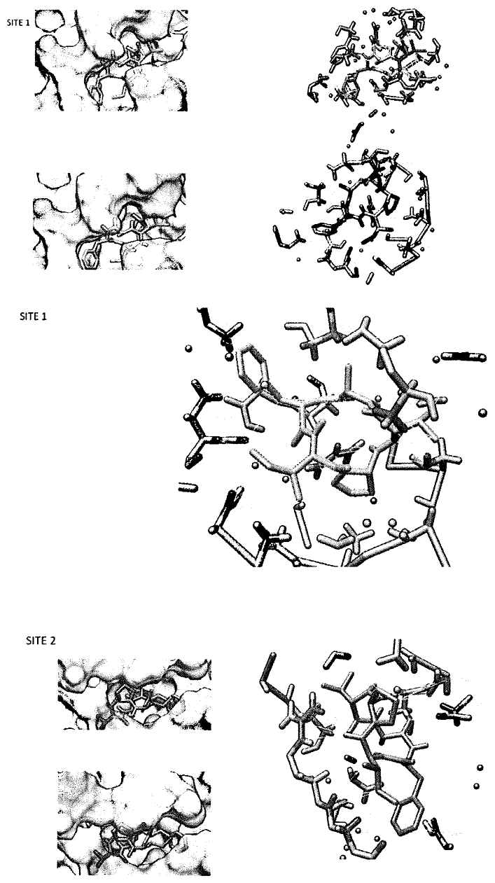

As discussed in the Examples and illustrated in FIG. 27, the inventors found that the DOS compounds likely bind in domain A of sestrin 2, at the interface with domain B and C and, importantly, far away from the double aspartate (D406/D407) GATOR/mTORC1 regulatory site (Kim et al., 2015). In fact, the biotinylated compound was found to bind to sMAC but not elements of the gator/mTORC1 complex in T cells (FIG. 8D). Accordingly, it is preferred that the cyclic polypeptide, derivative or analogue thereof is configured to bind to domain A of sestrin 2. Even more preferably, the cyclic polypeptide, derivative or analogue thereof is configured to bind to domain A of sestrin 2, and at the interface of domain B and domain C of sestrin 2. Preferably, the cyclic polypeptide, derivative or analogue thereof is configured to bind away from (i.e. not bind to) the double aspartate (D406/D407) GATOR/mTORC1 regulatory site of sestrin 2.

Additionally, it is expected that the DOS compounds will also bind in domain A of sestrin 1 and 3. Accordingly, it is preferred that the cyclic polypeptide, derivative or analogue thereof is configured to bind to domain A of sestrin 1 and/or sestrin 3. Even more preferably, the cyclic polypeptide, derivative or analogue thereof is configured to bind to domain A of sestrin 1 and/or sestrin 3, and at the interface of domain B and domain C of sestrin 1 and/or sestrin 3. Preferably, the cyclic polypeptide, derivative or analogue thereof is configured to bind away from (i.e. not bind to) the GATOR/mTORC1 regulatory site of sestrin 1 and/or sestrin 3. It is also noted that the regulatory site in sestrin1 and sestrin 3 may not be necessarily identical, especially in sestrin 3, as its interaction to GATOR was found to be constitutive (e.g. not sensitive to leucine deprivation) (Wolfson et al., 2016).

Preferably, the cyclic polypeptide, derivative or analogue thereof comprises or consists of between 2 and 20 amino acid residues, more preferably between 2 and 15 amino acid residues, and more preferably between 2 and 10 amino acid residues, and even more preferably between 2 and 7 amino acids. Preferably, the cyclic polypeptide, derivative or analogue thereof comprises or consists of between 3 and 20 amino acid residues, more preferably between 3 and 15 amino acid residues, and more preferably between 3 and amino acid residues, and even more preferably between 3 and 7 amino acids. Preferably, the cyclic polypeptide, derivative or analogue thereof comprises or consists of between 4 and 20 amino acid residues, more preferably between 4 and 15 amino acid residues, and more preferably between 4 and 10 amino acid residues, and even more preferably between 4 and 7 amino acids.

Preferably, the cyclic polypeptide, derivative or analogue thereof comprises or consists of between 2 and 15 amino acid residues, more preferably between 3 and 9 amino acid residues, and more preferably between 3 and 8 amino acid residues, and even more preferably between 4 and 7 amino acids.

Most preferably, the cyclic polypeptide, derivative or analogue thereof comprises or consists of 5 amino acid residues.

In one embodiment, the cyclic polypeptide, derivative or analogue thereof comprises or consists of an amino acid sequence as set out in any one of SEQ ID Nos: 8 to 84, as follows:

| SEQ | Amino | |
|---|---|---|
| ID | acid | Compound |
| No | sequence | name |
| 8 | MIVAD | DOS₁ |
| 9 | IVADS | DOS₂ |
| 10 | VADSE | DOS₃ |
| 11 | ADSEC | DOS₄ |
| 12 | DSECR | DOS₅ |
| 13 | SECRA | DOS6 |
| 14 | ECRAE | DOS₇ |
| 15 | CRAEL | DOS8 |
| 16 | RAELK | DOS₉ |
| 17 | LRFAP | DOS₁₆ |
| 18 | GDSGP | DOS₂₄ |
| 19 | QRESR | DOS₃₂ |
| 20 | RGPRG | DOS₃₉ |
| 21 | GPRGP | DOS₄₀ |
| 22 | AFIPV | DOS₄6 |
| 23 | IPVEE | DOS₄8 |
| 24 | EGAES | DOS₅6 |
| 25 | EQHLG | DOS6₂ |
| 26 | QHLGL | DOS6₃ |
| 27 | HLGLE | DOS6₄ |
| 28 | LGLEA | DOS6₅ |
| 29 | MSSGR | DOS₇₁ |
| 30 | SSGRV | DOS₇₂ |
| 31 | SGRVD | DOS₇₃ |
| 32 | NLAVV | DOS₇8 |
| 33 | VVMGL | DOS8₁ |
| 34 | VMGLH | DOS8₂ |
| 35 | HPDYF | DOS86 |
| 36 | PDYFT | DOS8₇ |
| 37 | DYFTS | DOS88 |
| 38 | FTSFW | DOS₉₀ |
| 39 | TSFWR | DOS₉₁ |
| 40 | WRLHY | DOS₉₄ |
| 41 | RLHYL | DOS₉₅ |
| 42 | LHYLL | DOS₉6 |
| 43 | HYLLL | DOS₉₇ |
| 44 | YLLLH | DOS₉8 |
| 45 | LLLHT | DOS₉₉ |
| 4⁶ | TDGPL | DOS₁₀₃ |
| 47 | DGPLA | DOS₁₀₄ |
| 4⁸ | GPLAS | DOS₁₀₅ |
| 49 | PLASS | DOS₁₀6 |
| 50 | LASSW | DOS₁₀₇ |
| 51 | ASSWR | DOS₁₀8 |
| 52 | SWRHY | DOS₁₁₀ |
| 53 | WRHYI | DOS₁₁₁ |
| 54 | RHYIA | DOS₁₁₂ |
| 55 | HYIAI | DOS₁₁₃ |
| 5⁶ | YIAIM | DOS₁₁₄ |
| 57 | AIMAA | DOS₁₁6 |
| 5⁸ | IMAAA | DOS₁₁₇ |
| 59 | MAAAR | DOS₁₁8 |
| 6₀ | AAARH | DOS₁₁₉ |
| 6₁ | AARHQ | DOS₁₂₀ |
| 6₂ | ARHQC | DOS₁₂₁ |
| 6₃ | RHQCS | DOS₁₂₂ |
| 6₄ | HQCSY | DOS₁₂₃ |
| 6₅ | QCSYL | DOS₁₂₄ |
| 66 | CSYLV | DOS₁₂₅ |
| 67 | SYLVG | DOS₁₂6 |
| 68 | YLVGS | DOS₁₂₇ |
| 69 | LVGSH | DOS₁₂8 |
| 70 | VGSHM | DOS₁₂₉ |
| 71 | HMAEF | DOS₁₃₂ |
| 72 | LQTGG | DOS₁₃₇ |
| 73 | GGDPE | DOS₁₄₀ |
| 74 | GDPEW | DOS₁₄₁ |
| 75 | DPEWL | DOS₁₄₂ |
| 7⁶ | PEWLL | DOS₁₄₃ |
| 77 | EWLLG | DOS₁₄₄ |
| 7⁸ | WLLGL | DOS₁₄₅ |
| 79 | RAPEK | DOS₁₅₁ |
| 8₀ | APEKL | DOS₁₅₂ |
| 8₁ | PEKLR | DOS₁₅₃ |
| 8₂ | EKLRK | DOS₁₅₄ |
| 8₃ | KLRKL | DOS₁₅₅ |
| 8₄ | LRKLS | DOS₁₅6 |

Accordingly, a preferred cyclic polypeptide, derivative or analogue thereof according to the invention comprises or consists of an amino acid sequence substantially as set out in any one of SEQ ID Nos: 8 to 84, or a functional variant or fragment thereof. The peptides of the first aspect are referred to herein as the DOS compounds, i.e. disruptors of sMAC.

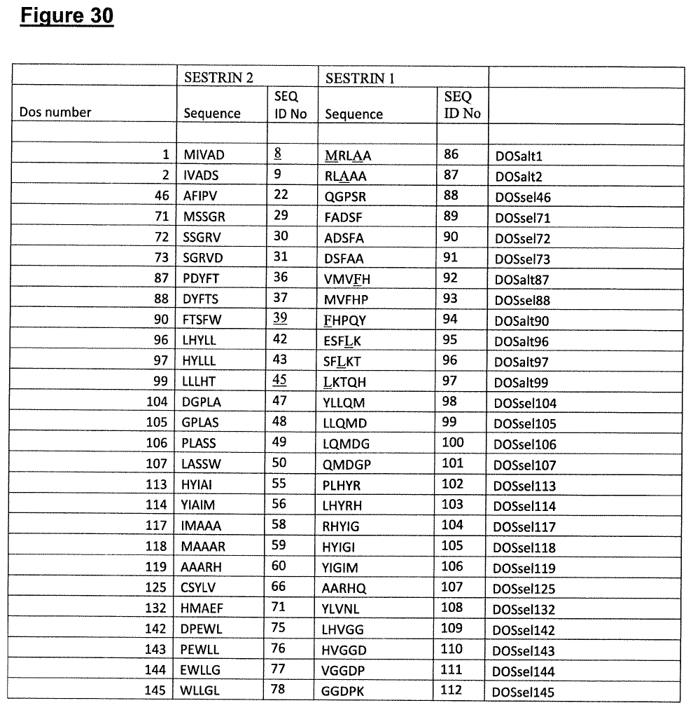

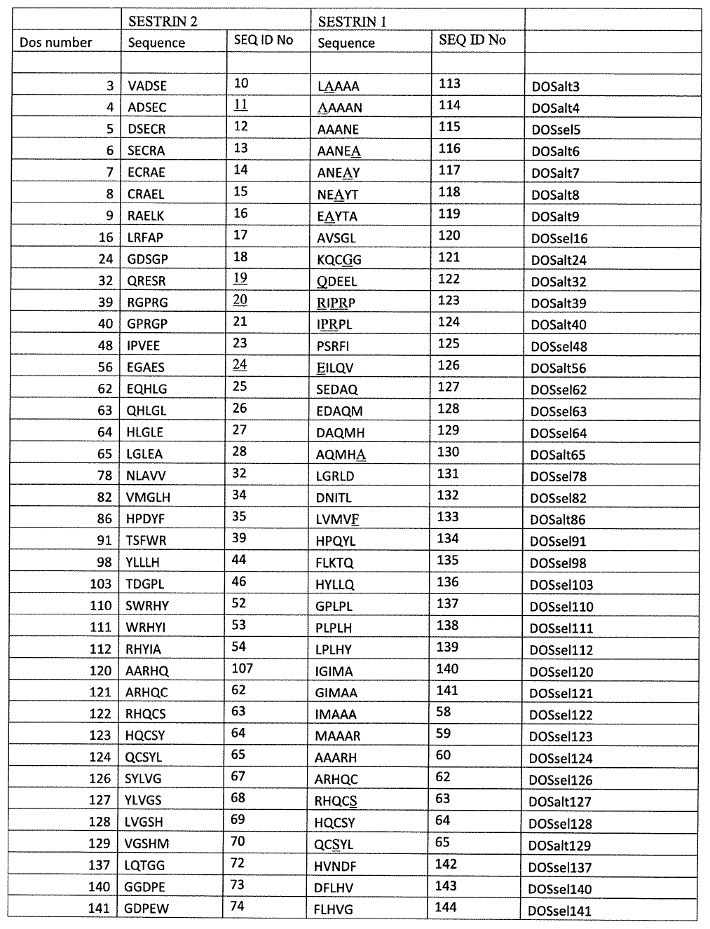

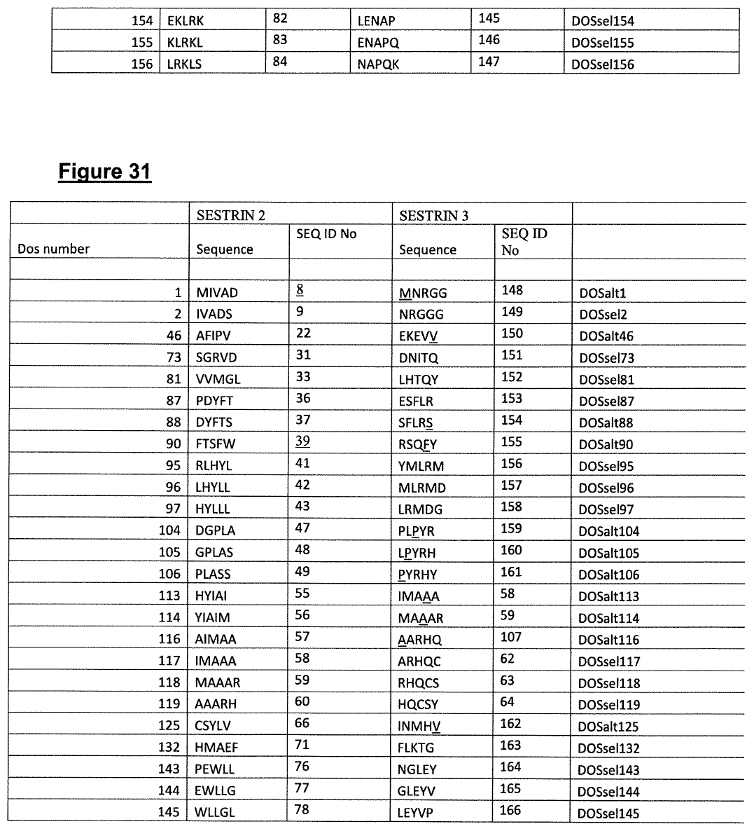

As illustrated in FIGS. 30 and 31, the cyclic polypeptide may be referred to as DOSₐₗₜ, i.e. cyclic polypeptides with conserved amino acid residues between sestrin 1/3 and sestrin 2 upon blast search in the same position. Alternatively, the cyclic polypeptide may be referred to as DOSₛₑₗ, i.e. cyclic polypeptides with no conserved amino acid residues between sestrin 1/3 and sestrin 2 upon blast search in the same position. It is noted that in circumstances where selective inhibition is preferred, a selective compound that is devoid of an essential motif may be desirable. For instance, SEQ ID No: 167 (DOSsel3***, RGGGS) can be a most preferred selective inhibitor for epilepsy, that is a condition where sestrin 3 plays a role in the disease pathogenesis (Johnson et al., 2015).

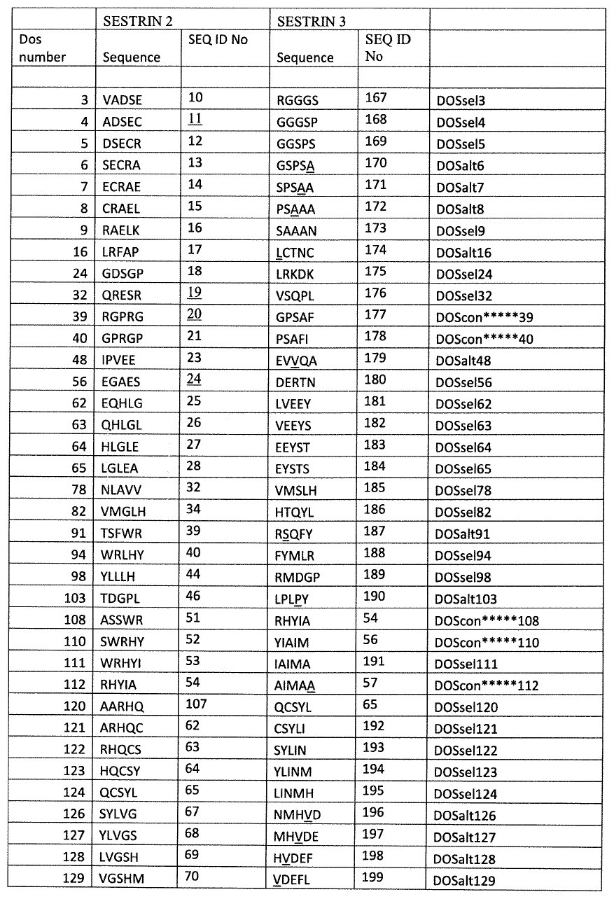

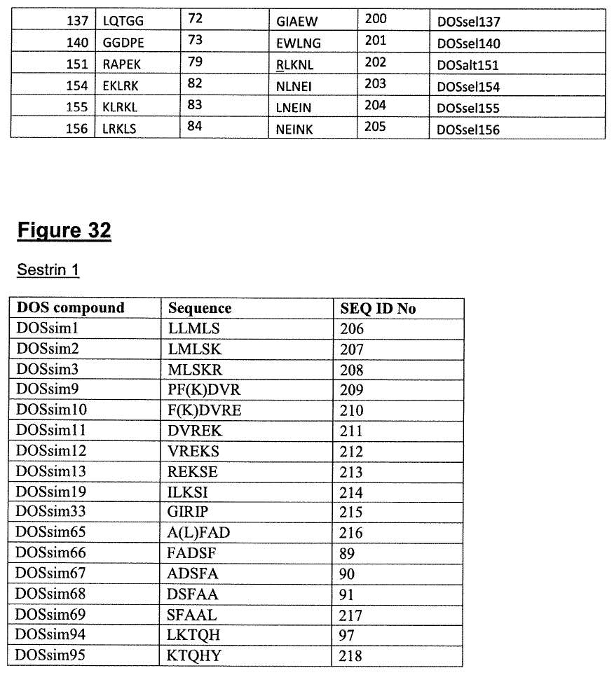

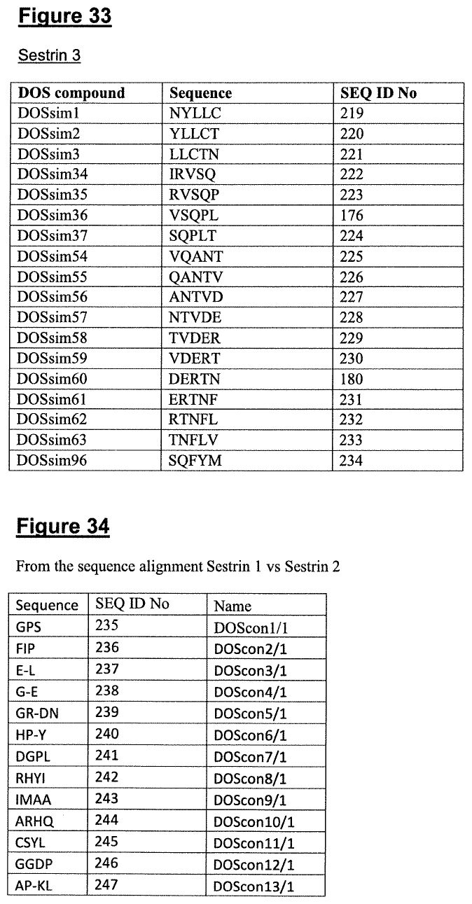

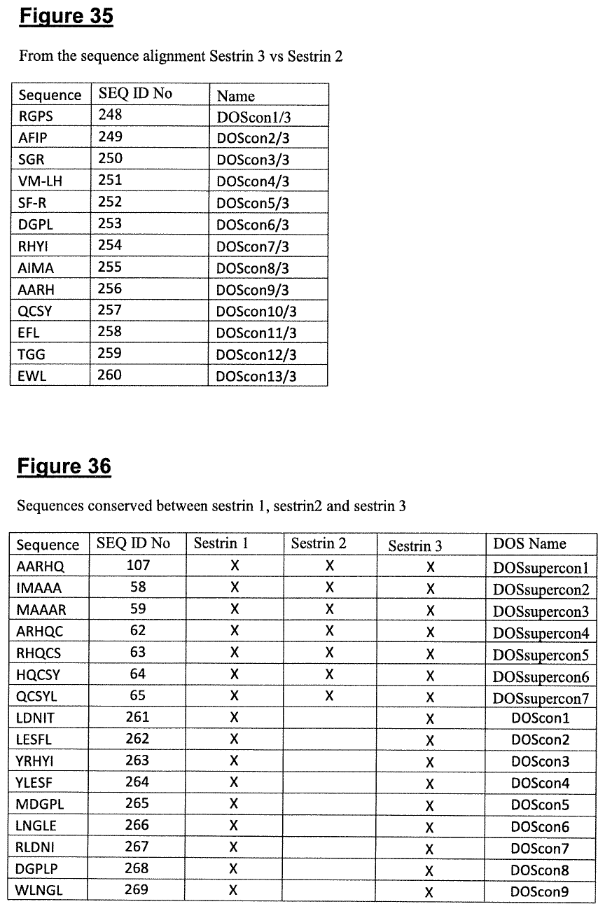

Alternatively, as shown in FIGS. 32 and 33, the cyclic polypeptide may be referred to as DOSₛᵢₘ, i.e. where a blast search identifies a region of homology (chemical similarities) in a different position. Furthermore, it is noted the existence of two types of consensus sequence; the first where the sequences overlap and/or have chemical similarities between any two sestrins and they are referred here as DOS_{con} (FIG. 34 and FIG. 35); the second type where the sequences completely overlap between all three sestrins. These are indicated as DOS_{supercon}. (FIG. 36).

Accordingly, in another embodiment, the cyclic polypeptide, derivative or analogue thereof according to the invention comprises or consists of an amino acid sequence substantially as set out in any one of SEQ ID Nos: 58, 59, 63, 65, 86, 87, 92, 94 to 97, 107, 113, 114, 116 to 119, 121 to 124, 126, 130, 133, 148, 150, 154, 155, 159 to 162, 170 to 172, 174, 179, 187, 190, 196 to 199, or 202, or a functional variant or fragment thereof. These cyclic peptides are referred to as DOSₐₗₜ cyclic polypeptides.

Alternatively, in yet another embodiment, the cyclic polypeptide, derivative or analogue thereof according to the invention comprises or consists of an amino acid sequence substantially as set out in any one of SEQ ID Nos: 58 to 60, 62 to 65, 71 to 73, 88 to 91, 93, 98 to 112, 115, 120, 125, 127 to 129, 131, 132, 134 to 147, 149, 151 to 153, 156 to 158, 163 to 169, 173, 175, 176, 180 to 186, 188, 189, 191 to 195, 200, 201, or 203 to 205, or a functional variant or fragment thereof. These cyclic peptides are referred to as DOSₛₑₗ cyclic polypeptides.

Alternatively, in yet another embodiment, the cyclic polypeptide, derivative or analogue thereof according to the invention comprises or consists of an amino acid sequence substantially as set out in any one of SEQ ID Nos: 89 to 91, 97, 176, 180, or 206 to 234, or a functional variant or fragment thereof. These cyclic peptides are referred to as DOSₛᵢₘ cyclic polypeptides.

Alternatively, in yet another embodiment, the cyclic polypeptide, derivative or analogue thereof according to the invention comprises or consists of an amino acid sequence substantially as set out in any one of SEQ ID Nos: 54, 56, 57, 177, 178, 235 to 269, or a functional variant or fragment thereof. These cyclic peptides are referred to as DOS_{con} cyclic polypeptides.

Alternatively, in yet another embodiment, the cyclic polypeptide, derivative or analogue thereof according to the invention comprises or consists of an amino acid sequence substantially as set out in any one of SEQ ID Nos: 58, 59, 62 to 65, 107, or a functional variant or fragment thereof. These cyclic peptides are referred to as DOS_{supercon} cyclic polypeptides.

It is well known to the skilled person that amino acids can be in the L-form or the D-form. Accordingly, in one embodiment the cyclic polypeptide, derivative or analogue thereof consists solely of L-amino acids. As such, it will be appreciated that in this embodiment, the cyclic polypeptide of the invention is an L-enantiomer. Alternatively, in another embodiment, the cyclic polypeptide, derivative or analogue thereof consists solely of D-amino acids. As such, in this embodiment, the cyclic polypeptide of the invention is a D-enantiomer. Alternatively, in another embodiment, the cyclic polypeptide, derivative or analogue thereof includes both D- and L-amino acids. The name given to the cyclic polypeptides of the invention indicates whether they are a D- or L-enantiomer, for example, DOS46L (SEQ ID No:22) is the L-enantiomer, whereas DOS46D is the D-enantiomer.

A most preferred cyclic polypeptide, derivative or analogue thereof comprises or consists of an amino sequence substantially as set out in SEQ ID No: 22 or a functional variant or fragment thereof. Even more preferably, the cyclic polypeptide, derivative or analogue thereof comprises an L-enantiomer or consists of L-amino sequences substantially as set out in SEQ ID No: 22. This peptide is referred to herein as DOS46L.

Another preferred cyclic polypeptide, derivative or analogue thereof comprises or consists of an amino sequence substantially as set out in SEQ ID No: 77, or a functional variant or fragment thereof. Even more preferably, the cyclic polypeptide, derivative or analogue thereof comprises a D-enantiomer or consists of D-amino sequences substantially as set out in SEQ ID No: 77. This peptide is referred to herein as DOS144D.

Another preferred cyclic polypeptide, derivative or analogue thereof comprise or consists of an amino sequence substantially as set out in SEQ ID No: 47, or a functional variant or fragment thereof. Even more preferably, the cyclic polypeptide, derivative or analogue thereof comprises an L-enantiomer or consists of L-amino sequences substantially as set out in SEQ ID No: 47. This peptide is referred to herein as DOS104L.

It will be appreciated that any of the polypeptides or peptides described herein may be synthesised de novo using standard peptide synthesis methods commonly known to the skilled person, and, as such, may then be used in any of the therapeutic applications described herein. Accordingly, any of the peptides can be produced by forming a peptide bond between adjacent amino acids of the sequences provided herein to build up to the full sequence length, i.e. the first amino acid is provided, to which a second amino acid is attached, and so on up to the desired length of peptide. Therefore, it is not necessary to start with the full sestrin 1, 2, or 3 sequence (preferably sestrin 2), or a shorter truncation thereof, and reduce the length of the polypeptide by removing amino acids from the N- and/or C-terminal until the desired peptide length is reached. Indeed, for the sake of speed, convenience and cost, it is preferred that the polypeptide, or biologically active variant or fragment thereof, is created using a de novo peptide synthesis method. Once the polypeptides or peptides have been synthesised, they can then undergo cyclisation.

It will be appreciated that any of the sequences represented as SEQ ID Nos: 8 to 84 can be readily cyclated to form a cyclic polypeptide, or derivative or analogue of the first aspect. For example, cyclization of peptides can be achieved by sidechain-to-sidechain, head-to-sidechain, tail-to-sidechain, head-to-tail (C-terminus to N-terminus), or backbone-to-backbone cyclization techniques. In one preferred embodiment, head-to-tail cyclization is the preferred method by which the cyclic polypeptides are produced. The cyclic polypeptides may be synthesised using either classical solution (liquid)-phase linear peptide cyclization or resin (solid)-based cyclization. In this embodiment, the polypeptide is produced using a cyclization cleavage approach, in which the cyclic polypeptide is synthesized by cyclization after step-wise linear peptide synthesis. An advantage of this method is that the sidechain does not need to be anchored, making the approach more general. Preferably, prior to use, resultant samples of cyclic peptides can be analysed by MALDI-TOF MS.

In a preferred embodiment, cyclisation is achieved through an amide bond. Alternatively, cyclisation may be achieved through an ester bond, or a disulphide bond.

As such, in a second aspect, there is provided a cyclic polypeptide, derivative or analogue thereof, comprising or consisting of an amino acid sequence selected from any of SEQ ID Nos: 8 to 84.

As described in the Examples, the inventors showed that the DOS compounds (for instance, DOS46L) derived from sestrin 2 (i.e. the polypeptide of the first aspect) bind to and inhibit the sestrin 1 and sestrin 3 proteins, as well sestrin 2 within the sMAC complex (FIG. 8D). In fact, the inventors discovered that some of the DOS compounds were conserved or had homology across all three sestrin proteins (sestrin 1, sestrin 2 and sestrin 3), that is expected to maintain the same binding/inhibitory properties. These compounds are referred to as pan inhibitors. However, the inventors also discovered that some of the DOS compounds were only able to specifically bind or inhibit one or two isoforms of sestrin, i.e. they are selective inhibitors.

However, it should be considered that direct binding is not a pre-requisite for DOS sestrin inhibitory function. For example, DOS46L does not directly bind to recombinant sestrin 3 in vitro yet it is found in a complex with sestrin 3 in real cells (most preferably T cells) with the entire sMAC, which results in pan-sestrin inhibition upon sMAC complex disruption (FIG. 8B-D).

Therefore, in one embodiment, the cyclic polypeptide, derivative or analogue thereof may bind to and/or inhibit sestrin 1, sestrin 2 and sestrin 3. In this embodiment, the cyclic polypeptide, derivative or analogue thereof is an inhibitor (or antagonist) of sestrin 1, sestrin 2 and sestrin 3. Alternatively, in another embodiment, the cyclic polypeptide, derivative or analogue thereof may selectively bind to and/or inhibit sestrin 1, sestrin 2 and/or sestrin 3. In this embodiment, the cyclic polypeptide, derivative or analogue thereof is a selective inhibitor of sestrin 1, sestrin 2 and/or sestrin 3.

In another embodiment, the cyclic polypeptide, derivative or analogue thereof may not bind to and/or inhibit sestrin 2, and selectively bind to and/or inhibit sestrin 1 and/or sestrin 3. In this embodiment, the cyclic polypeptide, derivative or analogue thereof is a selective inhibitor of sestrin 1 and/or sestrin 3.

In another embodiment, the cyclic polypeptide, derivative or analogue thereof may not bind to and/or inhibit sestrin 3, and selectively bind to and/or inhibit sestrin 1 and/or sestrin 2. In this embodiment, the cyclic polypeptide, derivative or analogue thereof is a selective inhibitor of sestrin 1 and/or sestrin 2.

In another embodiment, the cyclic polypeptide, derivative or analogue thereof may not bind to and/or inhibit sestrin 1, and selectively bind to and/or inhibit sestrin 2 and/or sestrin 3. In this embodiment, the cyclic polypeptide, derivative or analogue thereof is a selective inhibitor of sestrin 2 and/or sestrin 3.

In one embodiment, the cyclic polypeptide, derivative or analogue thereof is a selective inhibitor of sestrin 1, wherein the sestrin 1 has an amino acid sequence substantially as set out in SEQ ID No: 1, 2 or 3, or a variant or fragment thereof.

Preferably, in this embodiment, the cyclic polypeptide, derivative or analogue thereof is a selective inhibitor of sestrin 1, and comprises or consists of an amino acid sequence substantially as set out in SEQ ID Nos: 58 to 60, 62, 64, 88 to 91, 93, 98 to 112, 115, 120, 125, 127 to 129, 131, 132, or 134 to 147. Even more preferably, a selective inhibitor of sestrin 1 can be found in FIG. 30, indicated as DOSsel*. For instance, SEQ ID: 138 is a selective DOS inhibitor of sestrin 1. The sequence is PLPLH.

In another embodiment, the cyclic polypeptide, derivative or analogue thereof is a selective inhibitor of sestrin 3, wherein the sestrin 3 has an amino acid sequence substantially as set out in SEQ ID No: 5, 6 or 7, or a variant or fragment thereof.

Preferably, in this embodiment, the cyclic polypeptide, derivative or analogue thereof is a selective inhibitor of sestrin 3, and comprises or consists of an amino acid sequence substantially as set out in SEQ ID Nos: 47, 58 or 77, 62 to 65, 71 to 73, 149, 151 to 153, 156 to 158, 163 to 169, 173, 175, 176, 180 to 186, 188, 189, 191 to 195, 200, 201, or 203 to 205. Even more preferably, a selective inhibitor of sestrin 3 can be found in FIG. 31, indicated as DOSsel ***. For instance, SEQ ID: 167 is a selective DOS inhibitor of sestrin 3. The sequence is RGGGS.

The inventor has generated a general formula, as illustrated in FIG. 26.

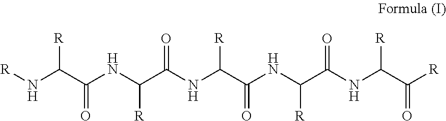

Hence, preferably, the cyclic polypeptide, derivative or analogue thereof of the invention comprises or consists of a cyclised form of formula (I).

It will be appreciated that the R groups of formula (I) are selected most preferably from standard amino acids well known to the skilled person. Accordingly, the R groups of formula (I) may be selected from any one of the following amino acids: alanine, cysteine, aspartic acid, glutamic acid, phenylalanine, glycine, histidine, isoleucine, lysine, leucine, methionine, asparagine, proline, glutamine, arginine, serine, threonine, valine, tryptophan and/or tyrosine. In addition it is observed the possibility that any amino acid in the general formula may be substituted with a non-natural variant that is known to the skilled in the art.

For the avoidance of doubt non-natural amino acids that may be used, but not limited to, include: 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4 Diaminobutyric acid, Desmosine, 2.2, Diaminopimelic acid, 2.3-Diaminopropionic acid, N-Ethylglycine, N-Ethylasparagine, Hydroxylysine, allo-Hydroxylysine, 3-Hydroxyproline, 4-Hydroxyproline, Isodesmosine, allo-Isoleucine, N-Methylglycine, sarcosine, N-Methylisoleucine, 6-N-Methyllysine, N-Methvlvaline, Norvaline, Norleucine, Ornithine.

In one embodiment, the cyclic polypeptide, derivative or analogue thereof comprising or consisting of formula (I), comprises, but is not limited to, binding or conjugation of:
(i) the terminal NH₂ and COOH groups (i.e. peptidic binding);
(ii) the NH₂ and NH₂ groups (i.e. hydrazine binding);
(iii) the NH₂ and CONH₂ groups (i.e. primary amidic binding);
(iv) the N═NH and NH₂ groups (i.e. hydrazine binding);
(v) the NH₂ and OH groups (i.e. hydroxylamine binding);
(vi) the NH₂ and SH groups (i.e. thiol-hydroxylamine binding);
(vii) the NH₂ and SR groups (i.e. hydrosulphide binding);
(viii) the COOH and CONH₂ groups (i.e. amide binding);
(ix) the COOH and COOH groups (i.e. dicarboxyl acid binding);
(x) the COOH and N═NH groups (i.e. carboxyl-amidogen binding);
(xi) the COOH and OH groups (i.e. hydroxy carboxylic binding);
(xii) the COOH and SH groups (i.e. thiol binding);
(xiii) the COOH and SR groups (i.e. thiol binding);
(xiv) the N═NH and CONH₂ groups (i.e. amidogen binding);
(xv) the N═NH and OH groups (i.e. N-hydroxylamine binding);
(xvi) the CONH₂ and CONH₂ groups (i.e. diamide binding);
(xvii) the CONH₂ and SR groups (i.e. s-amide binding);
(xviii) the SR and OH groups (i.e. hydroxy-thiol binding);
(xix) the SR and NH groups (i.e. thiol amine binding);
(xx) the NH₂ and NH groups (i.e. hydrazine binding);
(xxi) the COOH and NH groups (i.e. amino-propanoic binding);
(xxii) the SH and OH groups (i.e. hydrophilic binding);
(xxiii) the SH and NH groups (i.e. amino-thiol binding);
(xxiv) the OH and NH groups (i.e. hydroxylamine binding);
(xxv) the OH and OH groups (i.e. hydroxy binding);
(xxvi) the CONH₂ and OH groups (i.e. hydroxyamide binding);
(xxvii) the SH and SH groups (i.e. dithiol binding);
(xxviii) the CONH₂ and NH groups (i.e. hydroxyamide binding); or
(xxix) the NH and NH groups (i.e. diammonium binding), to create the cyclic polypeptide, derivative or analogue thereof.

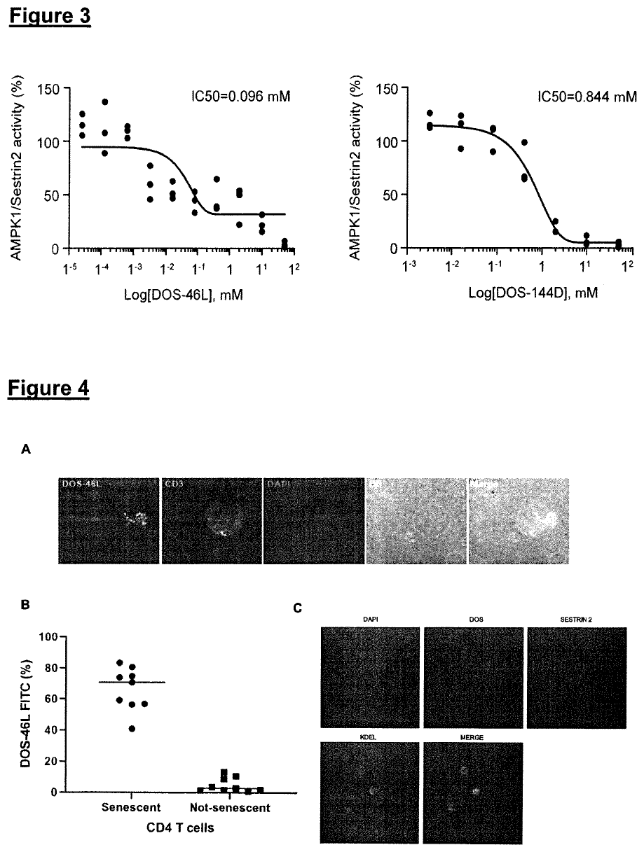

Any of the binding described above which differs from head to tail NH₂—COOH cyclisation may occur at any point of the molecule to generate DOSₐₗₜ (cyclic polypeptides with conserved amino acid residues between sestrin 1/3 and sestrin 2 upon blast search in the same position), DOSₛₑₗ (cyclic polypeptides with no conserved amino acid residues between sestrin 1/3 and sestrin 2 upon blast search in the same position), and DOSₛᵢₘ (where a blast search identifies a region of homology e.g. chemical similarities in a different position). Most preferably, cyclisation should not disrupt an essential amino acid in the sequence. For the avoidance of doubt, essential here means an amino acid that is conserved in at least one other sestrin protein upon a Blast search. It is also noted the existence of conserved motifs of at least 3 amino acids (not necessarily in continuation within the pentameric sequence) across any sestrin in a different location of the protein as result of frame shifting occurring during evolution and/or alternative splicing. For the avoidance of doubt the conserved motif should also be considered essential. For instance, the conserved motif of DOS46L is FIP while the conserved motif of DOS144D is WL-G. Comparing DOS46L vs DOS144D, the inventors discovered that head (A) to tail (V) cyclisation in DOS46L entails terminal non-essential A and V (alanine at the head and valine at the tail) resulting in better results over head to tail cyclisation in DOS144D where the head (E) to tail (G) cycle disrupts an essential amino acid (G, glycine at the tail) (FIG. 3). That result indicated the advantage of cyclizing against nonessential amino acids versus essential ones.

In one embodiment, the cyclic polypeptide, derivative or analogue thereof comprises or consists of a consensus or recurrentem sequence provided herein as SEQ ID No: 85, as follows:

[SEQ ID No: 85]
X₁X₂X₃X₄X₅

in which x₁, x₂, x₃, x₄ or x₅ is any amino acid residue. In one embodiment, x₁, x₂, x₃, x₄ or x₅ may be a non-polar and neutral amino acid, i.e. A, G, I, L, M, F, P or V.

In another embodiment, x₁, x₂, x₃, x₄ or x₅ may be a polar and neutral amino acid, i.e. C, S, T, Y, N or Q.

In another embodiment, x₁, x₂, x₃, x₄ or x₅ may be a polar and basic amino acid, i.e. H, K or R.

In another embodiment, x₁, x₂, x₃, x₄ or x₅ may be a polar and acidic amino acid, i.e. D or E.

Preferably, x₁ is A, P, H, L, R, M, D, F, V, I, E, N, Y, S, Q or T;
x₂ is Y, A, L, R, M, F, K, V, E, I, D, S, Q, N, W or T;
x₃ is L, A, S, R, M, D, E, K, F, T, Q, C, V, P, I or N;
x₄ is L, A, E, S, K, D, V, I, F, Q, H, C, T, P, N, R, or Y; and/or
x₅ is L, R, A, E, S, K, I, P, D, F, H, Y, C, T, N, Q, V, G or M.

Therefore, in one embodiment, the cyclic polypeptide, derivative or analogue thereof comprises or consists of an amino acid sequence as set out in SEQ ID No: 85, or a variant or fragment thereof.

Preferably SEQ ID No: 85, x₁ is A, P, H, L, R, M or D. Preferably, x₂ is Y, A, L or R. Preferably, x₃ is L, A, S or R. Preferably, x₄ is L, A or E. Preferably, x₅ is L, R, A or E. Preferably, cyclisation should not occur at amino acid within the consensus position. In the event that a cycle occurs between two or more amino acids within the consensus position, a software-based alignment may be used to determine which amino acid to be selected for cyclisation such that the one that is less recurrent should be preferred.

Beyond the consensus or recurrentem, and in addition to it, it is noted the presence of conserved motifs (consensus) as pointed above, most preferably of at least 3 amino acids (not necessarily in continuation) that are conserved in at least two sestrin proteins and account for pan sestrin inhibition upon targeting. That is regardless of their exact position in the protein (e.g. that could be present anywhere in the protein, albeit most favourably in the first 160 amino acids), result of frame shifting likely due to protein evolution and/or alternative splicing. As such, these motifs would be not be evident on a computer based search but they will manifest when frameshifting the DOS sequences starting from position one onwards (for instance, with a one amino acid pace). As noted above, for instance, this frameshifting allowed the inventors to discover the presence of the FIP and WL-G motifs within two of their preferred DOS compounds, DOS46L and DOS144D, respectively present in either sestrin1, sestrin 3, or both.

Accordingly, preferably, cyclization occurs at amino acid residues which are not essential for sestrin function. For example, amino acid residues that are not essential for sestrin function may be those that are not conserved in any of the three sestrins or within any of their conserved motif throughout the entire protein.

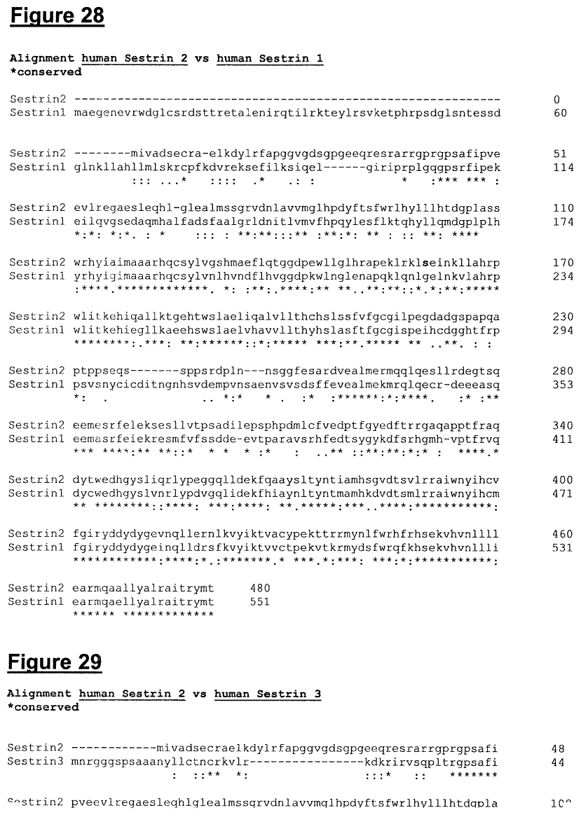

In another embodiment, considering the alignment between sestrin 2 and sestrin 1 (FIG. 28), cyclisation of the polypeptide, derivative or analogue thereof occur at amino acid residues 4, 9, 10, 15, 16, 19, 20, 21, 24, 26, 27, 28, 29, 30, 31, 32, 33, 34, 35, 36, 37, 39, 40, 41, 46, 50, 56, 61, 63, 65, 66, 67, 71, 73, 84, 91, 94, 96, 99, 103, 108, 109, 110, 131, 134, 147, 185, 204, 217, 220, 226, 228, 230, 233, 234, 236, 237, 238, 239, 240, 241, 244, 249, 251, 252, 255, 256, 272, 273, 274, 277, 284, 292, 298, 299, 301, 303, 304, 306, 309, 310, 311, 313, 314, 319, 331, 333, 334, 343, 359, 369, 431, 442, or 467 of sestrin 2 (SEQ ID No: 4), and/or amino acid residues 1-68, 72, 77, 78, 79, 84, 85, 88, 89, 90, 93, 95, 96, 97, 98, 99, 100, 102, 103, 104, 109, 113, 119, 124, 126, 128, 129, 130, 131, 135, 137, 148, 155, 158, 160, 163, 167, 172, 173, 174, 195, 198, 211, 249, 268, 281, 284, 290, 292, 294, 297, 298, 300 to 312, 315, 319, 320, 321, 323, 325, 326, 329, 330, 346, 347, 350, 357, 365, 371, 372, 374, 376, 378, 381, 382, 383, 385, 386, 391, 403, 405, 414, 430, 440, 502, 513, or 538 of sestrin 1 (SEQ ID No: 1).

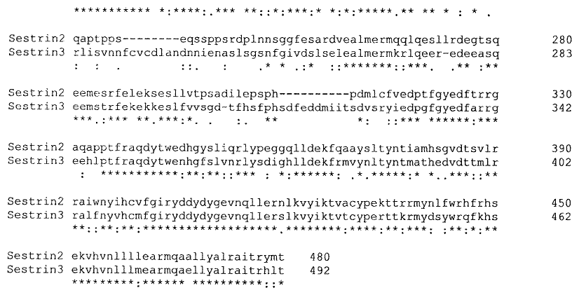

In another embodiment, considering the alignment between sestrin 2 and sestrin 3 (FIG. 29), cyclisation of the polypeptide, derivative or analogue thereof occurs at amino acid resides 1, 2, 4, 5, 10, 11, 14 to 29, 34, 35, 36, 39, 40, 41, 50, 56, 59, 61, 63, 66, 69, 70, 71, 81, 87, 91, 94, 96, 99, 103, 108, 109, 110, 131, 134, 142, 143, 147, 151, 180, 192, 204, 217, 220, 222, 224, 226, 228, 230, 231, 233, 234, 239, 240, 241, 243, 244, 245, 246, 249, 251, 255, 256, 272, 273, 277, 289, 299, 301, 302, 303, 306, 307, 310, 313, 314, 320, 331, 333, 334, 357, 359, 369, 380, 342, or 367 of sestrin 2 (SEQ ID No: 4), and/or amino acid residues 1 to 14, 16, 17, 22, 23, 30, 31, 32, 35, 36, 37, 46, 52, 55, 57, 59, 62, 65, 66, 67, 77, 83, 87, 90, 92, 95, 99, 104, 105, 106, 127, 130, 138, 138, 143, 147, 176, 188, 200, 213, 216, 218, 220, 222, 224, 226, 227, 229, 230, 232, 234, 235, 236, 237, 238, 239, 243, 244, 245, 247, 248, 249, 250, 253, 255, 259, 260, 276, 280, 292, 302, 304, 305, 308, 309, 312, 313, 314, 315, 316, 317, 318, 319, 320, 321, 322, 325, 326, 332, 343, 345, 346, 369, 371, 381, 392, 454, or 479 of sestrin 3 (SEQ ID No: 5).

Preferably, the cyclic polypeptide, derivative or analogue thereof targets sestrin in immune cells. More preferably, the cyclic polypeptide, derivative or analogue thereof targets sestrin in B cells, Natural Killer (NK) cells, and/or T cells (T lymphocytes). Even more preferably, the cyclic polypeptide, derivative or analogue thereof targets sestrin in CD4+ and/or CD8+ T cells. Even more preferably, the cyclic polypeptide, derivative or analogue thereof targets sestrin in CD4+ CD27+ CD28+ cells and/or CD4+ CD27− CD28+ cells. Most preferably, the cyclic polypeptide, derivative or analogue thereof targets sestrin in CD4+ CD27− CD28− T cells (i.e. senescent T cells).

Preferably, the cyclic polypeptide, derivative or analogue thereof induces or restores cytokine production. For example, preferably the cyclic polypeptide, derivative or analogue thereof induces or restores the production of IFN gamma, TNF alpha, IL-10 and/or IL-4. Preferably, the cyclic polypeptide, derivative or analogue thereof induces or restores cytokine production in immune cells, even more preferably in T cells. Most preferably, the T cells are senescent T cells.

Preferably, the cyclic polypeptide, derivative or analogue thereof prevents or reverses immune-senescence.

Immune-senescence is the gradual deterioration of the immune system brought on naturally by aging (Montecino-Rodriguez et al., 2013). Immune-senescence impacts both the host's capacity to respond to infections, and the development of long-term immune memory, especially by vaccination (Goronzy and Weyand, 2013). Immune-senescence is considered to be a major contributory factor to the increased frequency of morbidity and/or mortality among the elderly. Immune-senescence is triggered by the age-dependent accumulation of senescent lymphocytes, especially senescent T cells, with short telomeres, endogenous DNA damage, lack of telomerase activity or loss of telomere transfer from antigen presenting cells to T cells as recently discovered by the inventor (Lanna (Group), 2020), and loss of responsiveness to antigen stimulation, caused by increased sestrin expression. The compounds can restore both telomere transfer from antigen presenting cells to T cells that result in telomere elongation in the telomere acquiring T cells as shown in FIG. 23. Accordingly, preferably, the cyclic polypeptide, derivative or analogue thereof according to the invention, binds to and inhibits sestrin, resulting in the prevention of immune-senescence.

Preferably, the cyclic polypeptide, derivative or analogue thereof is configured to rejuvenate the immune system.

“Rejuvenating the immune system” can mean restoring immune cell responsiveness, especially T cells, to antigen stimulation. Additionally, rejuvenating the immune system can mean restoring telomere length and/or transfer, and/or telomerase activity in immune cells. Rejuvenating the immune system can also mean restoring antigen-specific proliferation and/or cytokine production in, immune cells, preferably T cells. In addition, rejuvenation can mean restoring antigen-specific antibody production by B cells and/or CD8⁺ T cells and/or Natural Killer cell expansion.

As described in the Examples, the inventors have generated novel cyclic polypeptides, derivatives or analogues of the invention (i.e. DOS compounds), which can revive immunity during aging with a single dose. For example, the inventors have found that a single injection of their cyclic peptides (DOS) restored anti-viral responses in old mice, therefore surprisingly demonstrating that DOS can be added to next-generation vaccine formulations. Additionally, the inventors have shown that the DOS compounds reduced senescent T cells, while increasing the levels of stem-like long lived memory T cells (Gattinoni et al., 2009, 2011). As such the compounds can be used to induce a stem like state or increase stemness in cells, including bone marrow resident cells for transplantation (Copelan, 2006). The DOS compounds also increased the levels of cytokines, such as IFN gamma, TNF alpha and IL-10 upon DOS treatment, and can therefore be used to prevent, ameliorate or treat a number of age-related conditions, or they act as immune-stimulators in an antigen specific fashion as shown in FIG. 25.

Thus, in a third aspect of the invention, there is provided the cyclic polypeptide, derivative or analogue thereof according to the first or second aspect, for use in therapy.

In a fourth aspect of the invention, there is provided a nucleic acid encoding the cyclic polypeptide, derivative or analogue thereof according to the first or second aspect, for use in therapy.

The nucleic acid molecule may be an isolated or purified nucleic acid sequence. The nucleic acid sequence may be a DNA sequence. The nucleic acid molecule may comprise synthetic DNA. The nucleic acid molecule may comprise cDNA. The nucleic acid may be operably linked to a heterologous promoter. The nucleic acid sequence encoding the cyclic polypeptide, derivative or analogue thereof, may be incorporated into a genetic construct for use in therapy or for cloning purposes.

In one preferred embodiment, therefore, the nucleic acid molecule encoding the cyclic polypeptide, derivative or analogue thereof is a genetic construct. More preferably, the nucleic acid molecule or genetic construct is provided in a recombinant vector.

Genetic constructs of the invention may be in the form of an expression cassette, which may be suitable for expression of the encoded cyclic polypeptide, derivative or analogue thereof, in a host cell. The genetic construct may be introduced into a host cell without it being incorporated in a vector. For instance, the genetic construct, which may be a nucleic acid molecule, may be incorporated within a liposome or a virus particle.

Alternatively, a purified nucleic acid molecule (e.g. histone-free DNA, or naked DNA) may be inserted directly into a host cell by suitable means, e.g. direct endocytotic uptake. For cloning purposes, the construct may be introduced directly into cells of a host subject (e.g. a bacterial, eukaryotic or animal cell) by transfection, infection, electroporation, microinjection, cell fusion, protoplast fusion or ballistic bombardment.

Alternatively, genetic constructs of the invention may be introduced directly into a host cell using a particle gun. Alternatively, the genetic construct may be harboured within a recombinant vector, for expression in a suitable host cell. For administration to the subject being treated, the construct may be contained in a phage delivery system, such as AAV.

The recombinant vector may be a plasmid, cosmid or phage. Such recombinant vectors are useful for transforming host cells with the genetic construct, and for replicating the expression cassette therein. The skilled technician will appreciate that genetic constructs of the invention may be combined with many types of backbone vector for expression purposes. Recombinant vectors may include a variety of other functional elements including a suitable promoter to initiate gene expression. For instance, the recombinant vector may be designed such that it autonomously replicates in the cytosol of the host cell. In this case, elements, which induce or regulate DNA replication, may be required in the recombinant vector. Alternatively, the recombinant vector may be designed such that it integrates into the genome of a host cell. In this case, DNA sequences which favour targeted integration (e.g. by homologous recombination) are envisaged.

To facilitate cloning, the recombinant vector may also comprise DNA coding for a gene that may be used as a selectable marker in the cloning process, i.e. to enable selection of cells that have been transfected or transformed, and to enable the selection of cells harbouring vectors incorporating heterologous DNA. Alternatively, the selectable marker gene may be in a different vector to be used simultaneously with vector containing the gene of interest. The vector may also comprise DNA involved with regulating expression of the coding sequence, or for targeting the expressed cyclic polypeptide to a certain part of the host cell.

In a fifth aspect, there is provided the cyclic polypeptide, derivative or analogue thereof according to the first or second aspect, or a nucleic acid encoding the cyclic polypeptide, derivative or analogue thereof, for use in treating, preventing or ameliorating a microbial infection, an age-related condition, or a T-cell mediated disease.

In a sixth aspect, there is provided a method of treating, preventing or ameliorating a microbial infection, an age-related condition, or a T-cell mediated disease in a subject, the method comprising, administering to a subject in need of such treatment, a therapeutically effect amount of the cyclic polypeptide, derivative or analogue thereof according to the first or second aspect, or a nucleic acid encoding the cyclic polypeptide, derivative or analogue thereof.

The microbial infection may be bacterial, viral, fungal or protist.

An age-related condition is a condition associated with, caused by, or related to, the aging and/or decline of the immune system. Preferably, the age-related condition is characterised by sestrin-dependent sMAC activation and/or immune-senescence (Lanna et al., 2017). For example, the age-related condition may be selected from a group consisting of cancer (e.g. paediatric neuroblastoma, melanoma, liver, pancreatic, testis or pancreatic cancer (Guo et al., 2020; Kumar et al., 2018; Wang et al., 2018; Won et al., 2019; Zhao et al., 2017)), smoke-induced emphysema (Heidler et al., 2013), epilepsy (Johnson et al., 2015), depression (Kato et al., 2019), metabolic diseases (EL-Ashmawy and Ahmed, 2019), osteoporosis (Oh et al., 2021), or neurodegenerative diseases, such as Alzheimer's disease (Rai et al., 2016) or dementia (Chen et al., 2019), as well erectile dysfunction, sterility and polycystic ovarian syndrome (Xu et al., 2021).

A T-cell mediated disease may be a disease selected from a group consisting of an infectious disease (e.g. a microbial infection), cancer, an autoimmune disorder (e.g. including type I diabetes), an inflammatory disorder, skin disorders including various injuries (chemical, electrical, physical), ulcers and burns.

As illustrated in the Examples and FIG. 5 in particular, some DOS compounds have the ability to inhibit all three sestrins (e.g. DOS46L and DOS144D) and therefore, can be defined as pan-inhibitors. Others inhibit only one or two sestrins (e.g. DOS104D and DOS117L) and therefore, can be defined as selective inhibitors. Accordingly, this demonstrates the possibility of selecting either a pan or selective inhibitor to inactivate sMAC, depending on the condition to be treated. For instance, a certain therapeutic approach may benefit from pan-inhibition of sestrins (e.g. by DOS46L), such as vaccination against bacterial or viral infections, whereas other conditions, such as epilepsy, are controlled by sestrin 3 (Johnson et al., 2015) and may benefit from a selective sestrin 3 inhibitor, such as DOS117L.

Accordingly, in one embodiment, there is provided the cyclic polypeptide, derivative or analogue thereof, comprising or consisting of an amino acid sequence substantially as set out in SEQ ID No: 47, 58 or 77, 62 to 65, 71 to 73, 149, 151 to 153, 156 to 158, 163 to 169, 173, 175, 176, 180 to 186, 188, 189, 191 to 195, 200, 201, or 203 to 205, for use in treating, preventing or ameliorating epilepsy. It will be appreciated that these peptides inhibit sestrin 3.

In another embodiment, there is provided the cyclic polypeptide, derivative or analogue thereof, comprising or consisting of an amino acid sequence substantially as set out in SEQ ID No: 8, 13, 14, 15, 38, 68 or 70 for use in treating, preventing or ameliorating a microbial infection. It will be appreciated that these peptides inhibit each of sestrin 1, 2 and 3.

In a seventh aspect, there is provided a composition comprising the cyclic polypeptide, derivative or analogue thereof of the first or second aspect, or a nucleic acid encoding the cyclic polypeptide, derivative or analogue thereof, and a vaccine.

In an eighth aspect, there is provided the cyclic polypeptide, derivative or analogue thereof of the first or second aspect, or a nucleic acid encoding the cyclic polypeptide, derivative or analogue thereof, for use in stimulating an immune response, preferably as a vaccine booster.

The vaccine may be any anti-microbial vaccine, such as an antiviral, antibacterial, antifungal or anti-protist vaccine.

Preferably, the cyclic polypeptide, derivative or analogue thereof of the first or second aspect is for use in stimulating a T-cell mediated immune response.

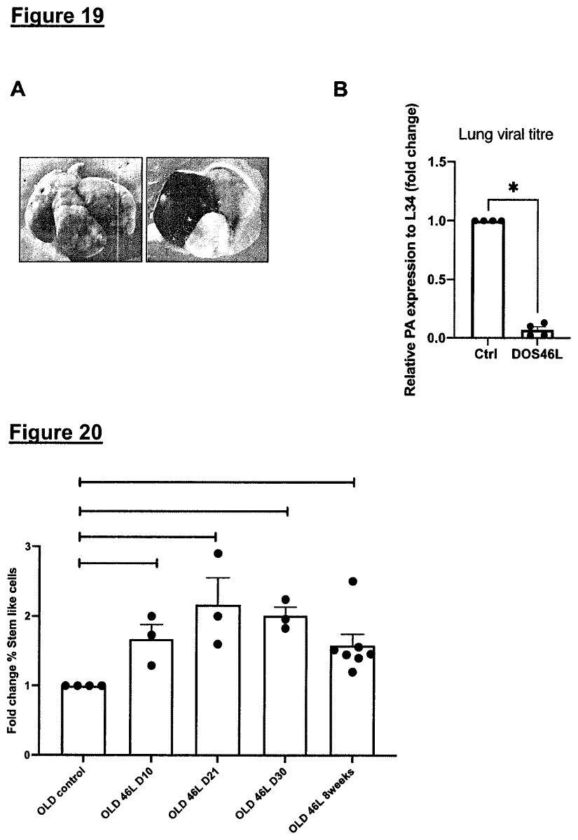

It will be appreciated that the cyclic polypeptide, derivative or analogue thereof according to the invention may be used in a medicament which may be used in a monotherapy (i.e. use of the cyclic polypeptide, derivative or analogue thereof), for treating, ameliorating, or preventing a microbial (e.g. bacterial or viral) infection, or an age-related condition. For example, the monotherapy use of DOS (e.g. DOS46L) was demonstrated after single dose administration in FIG. 18. In this example, a single injection of DOS prevented death of animal upon a lethal challenge of viral infection (H1N1 PR8/54). Notably, the effect is sustained in the long term (e.g. protection upon viral challenge 8 weeks after DOS administration sub-cutaneously).

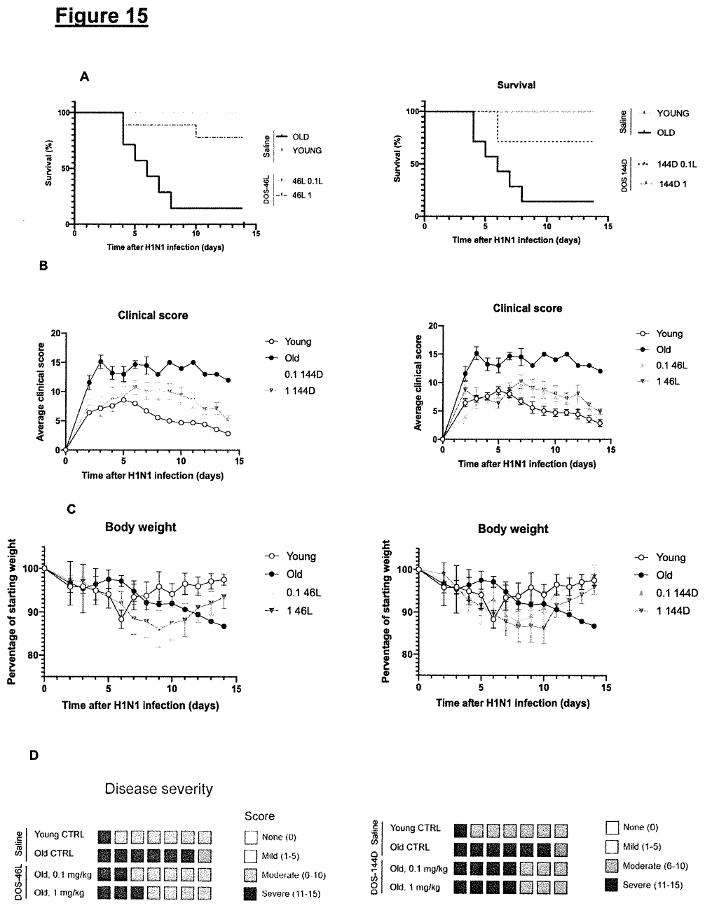

Alternatively, the cyclic polypeptide, derivative or analogue thereof according to the invention may be used as an adjunct to, or in combination with, known therapies for treating, ameliorating, or preventing microbial (e.g. bacterial or viral) infections, such as vaccine formulations for preventing infections, such as coronavirus or influenza. For example, the combinational use of DOS (e.g. DOS46L or DOS144D) was demonstrated after single dose administration along with a flu vaccine (FLUAD) in FIG. 15. As such, the cyclic polypeptide, derivative or analogue thereof can be administered with the vaccine formulation, so that the immune cells can be rejuvenated.

Alternatively, the cyclic polypeptide, derivative or analogue thereof according to the invention may be used as an adjunct to, or in combination with, known therapies for treating, ameliorating, or preventing age-related conditions. For example, the cyclic polypeptide, derivative or analogue may be used in combination with cancer immune-therapy drugs, such as checkpoint inhibitors.

The cyclic polypeptide according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising cyclic polypeptides according to the invention may be used in a number of ways. For instance, oral administration may be required, in which case the cyclic polypeptide may be contained within a composition that may, for example, be ingested orally in the form of a tablet, capsule or liquid. Alternatively, the cyclic polypeptide may be modified, for example, by methylation, to favour oral administration by providing resistance to proteolytic degradation. An alternative option for administrating the cyclic polypeptide would be to use a nasal spray. Hence, compositions comprising cyclic polypeptides of the invention may be administered by inhalation (e.g. intranasally). Compositions may also be formulated for topical use. For instance, creams or ointments may be applied to the skin.

Cyclic polypeptides according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with cyclic polypeptides used according to the invention is required and which would normally require frequent administration (e.g. at least daily injection).

In a preferred embodiment, medicaments according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the cyclic polypeptide, analogue or derivative thereof that is required is determined by its biological activity and bioavailability, which in turn depends on the mode of administration, the physiochemical properties of the cyclic polypeptide and whether it is being used as a monotherapy or in a combined therapy. The frequency of administration will also be influenced by the half-life of the cyclic polypeptide within the subject being treated. Optimal dosages to be administered may be determined by those skilled in the art, and will vary with the particular cyclic polypeptide in use, the strength of the pharmaceutical composition, the mode of administration, and the advancement of the condition to be treated. Additional factors depending on the particular subject being treated will result in a need to adjust dosages, including subject age, weight, gender, diet, and time of administration.

Generally, a daily dose of between 0.001 μg/kg of body weight and 10 mg/kg of body weight of the cyclic polypeptide according to the invention may be used for treating, ameliorating, or preventing a bacterial or viral infection, or an age-related condition, depending upon which cyclic polypeptide is used. More preferably, the daily dose is between 0.01 μg/kg of body weight and 1 mg/kg of body weight, and even more preferably between approximately 0.1 μg/kg and 100 μg/kg body weight, and most preferably between approximately 0.1 μg/kg and 10 μg/kg body weight.

The cyclic polypeptide may be administered before, during or after onset of the bacterial or viral infection, or the age-related condition. Daily doses may be given as a single administration (e.g. a single daily injection or inhalation of a nasal spray). Alternatively, the cyclic polypeptide may require administration twice or more times during a day. As an example, cyclic polypeptides may be administered as two (or more depending upon the severity of the bacterial or viral infection, or the age-related condition being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of cyclic polypeptide according to the invention to a patient without the need to administer repeated doses.

Known procedures, such as those conventionally employed by the pharmaceutical industry (e.g. in vivo experimentation, clinical trials, etc.), may be used to form specific formulations of the cyclic polypeptide according to the invention and precise therapeutic regimes (such as daily doses of the agents and the frequency of administration). The inventors believe that they are the first to suggest an anti-sMAC composition, based on the use of a cyclic polypeptide of the invention.

Hence, in a ninth aspect of the invention, there is provided a pharmaceutical composition comprising a therapeutically effective amount of the cyclic polypeptide, derivative or analogue thereof according to the first or second aspect, or a nucleic acid encoding the cyclic polypeptide, derivative or analogue thereof, and optionally a pharmaceutically acceptable vehicle.

The invention also provides in a tenth aspect, a process for making the pharmaceutical composition according to the ninth aspect, the process comprising combining a therapeutically effective amount of the cyclic polypeptide, derivative or analogue thereof according to the first or second aspect, or a nucleic acid encoding the cyclic polypeptide, derivative or analogue thereof, with a pharmaceutically acceptable vehicle.

A “subject” may be a vertebrate, mammal, or domestic animal. Hence, medicaments according to the invention may be used to treat any mammal, for example livestock (e.g. a horse), pets, or may be used in other veterinary applications. Most preferably, however, the subject is a human being. Even more preferably, the subject is at least 50, 60, 70, 80, 90 or 100 years old.

A “therapeutically effective amount” of cyclic polypeptide is any amount which, when administered to a subject, is the amount of active agent that is needed to treat the microbial (e.g. bacterial or viral or protist or parasite infection), or the age-related condition, or produce the desired effect.

For example, the therapeutically effective amount of cyclic polypeptide used may be from about 0.001 mg to about 800 mg, and preferably from about 0.01 mg to about 500 mg. It is preferred that the amount of cyclic polypeptide is an amount from about 0.1 mg to about 100 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, coatings, or tablet-disintegrating agents. The vehicle may also be an encapsulating material. In powders, the vehicle is a finely divided solid that is in admixture with the finely divided active agents according to the invention. In tablets, the active agent (i.e. the modulator) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention (the cyclic polypeptide) may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The cyclic polypeptide may be prepared as a sterile solid composition that may be dissolved or suspended at the time of administration using sterile water, saline, or other appropriate sterile injectable medium.

The cyclic polypeptide and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The cyclic polypeptide used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. It is also noted that the presence of conserved motives of at least three amino acids that can be found at a different location in the three sestrins is likely the result of protein sequence evolution and/or alternative splicing. For example, FIP is the conserved domain of DOS46L that is found in both sestrin 1 and 3, but at slightly different locations that still results in potent pan sestrin inhibition. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the sequence identified as SEQ ID Nos: 1-235, and so on.

Amino acid/polynucleotide/polypeptide sequences with a sequence identity which is greater than 60%, preferably greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in SEQ ID No: 1-235.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids. For instance, that been taken into consideration to generate a consensus and DOSₛᵢₘ compounds in FIG. 32-33.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 shows a model of the sestrin-MAPK complex (sMAC) in senescent T cells.

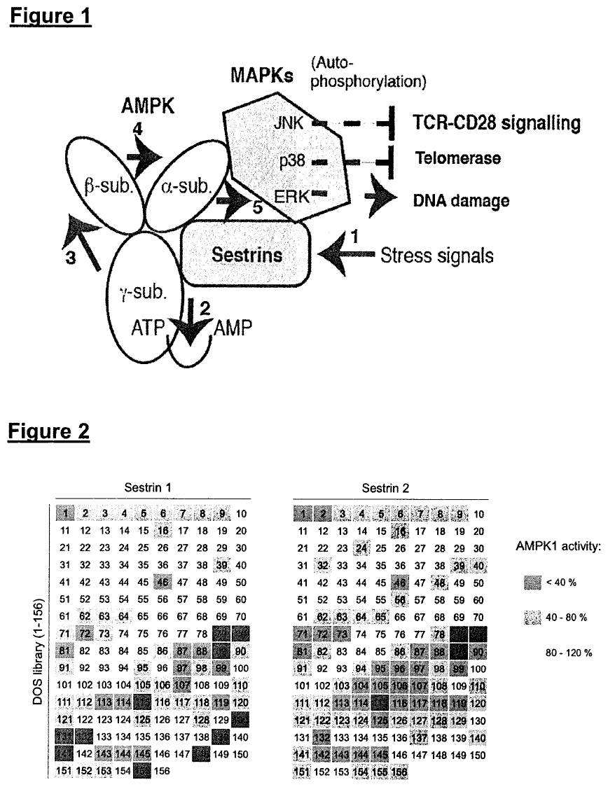

FIG. 2 demonstrates the inhibitory activities of various embodiments of the polypeptides of the invention (i.e. referred to herein as DOS peptides) as shown by the percentage of AMPK1 activity. DOS peptide inhibitory activities were screened by ATP consumption in an AMPK kinase reaction assay, in the presence of recombinant Sestrin1 and Sestrin2.

FIG. 3 illustrates the potency of polypeptides of the invention (DOS compounds). IC50 doses were calculated for the compounds that showed significant Sestrin 2 inhibition. Log concentration values are shown.

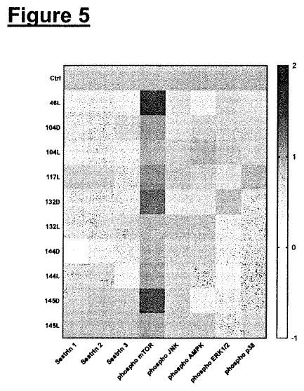

FIG. 4 illustrates the cell penetrability of DOS in T cells, and its location to sMAC. (A-B) CD4+ T cells were isolated from peripheral blood mononuclear cells (PBMCs) from healthy donors and treated with DOS compounds. (A) Representative confocal images of CD4+ T cells stained with DOS-FITC and CD3-AF594. (B) Quantification of DOS46L penetration by flow cytometry in CD4+ CD27-CD28-cells (Tsen) and in CD4+CD27+CD28+ (Terl). (C) Localisation of DOS in the ER (KDEL ER marker) where the sMAC resides. **p<0.01.

FIG. 5 shows a heat-map illustrating signal disruption by DOS compounds. CD4+ T cells were isolated from PBMCs from healthy donors, treated with DOS compounds for 4 hours and harvested for immunoblot analysis. The heat-map of Sestrin 1, Sestrin 2, Sestrin 3, p-mTOR1, p-JNK, p-AMPK, p-ERK1/2, and p-p38, represents the mean of three to nine experiments. Histone 3 was used as a control.

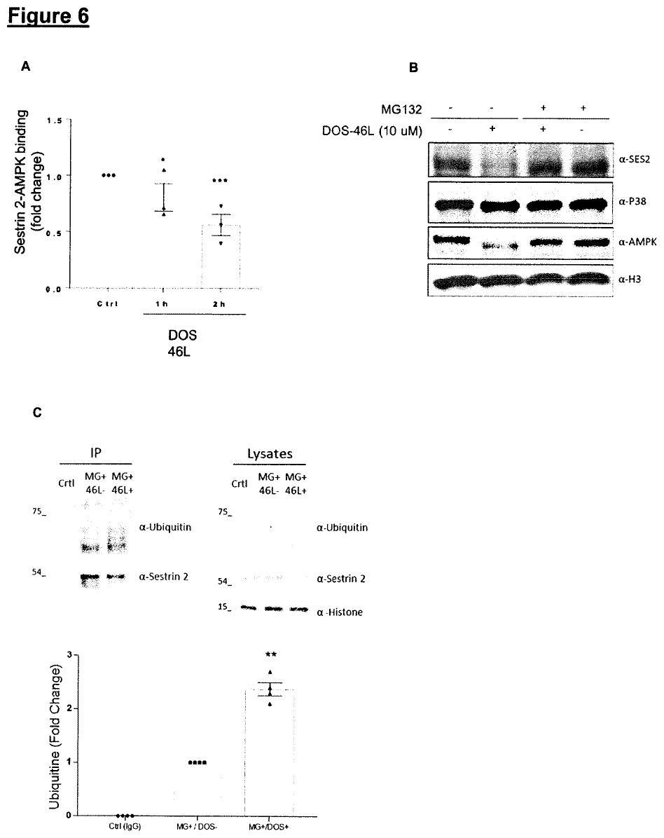

FIG. 6 illustrates sMAC disruption by DOS46L. (A) CD4+ T cells were purified from PBMCs from healthy donors and treated with DOS46L (10 uM) for the indicated time points. The binding of AMPK to Sestrin 2 was quantified against the total level of Sestrin 2 immunoprecipitated and detected by ELISA. IgG immunoprecipitates served as background control. (B) Cells were pre-treated with proteasome-inhibitor MG-132 (10 uM) for 30 min and subsequently treated with DOS46L (10 uM) for 4 hours. Cells were harvested for immunoblot analysis or proceeded to immunoprecipitation. Levels of Sestrin 2, p38, and AMPK were evaluated by immunoblotting. H3 was used as a loading control. Representative images of 3 experiments are shown. (C) Immunoblot levels of Ubiquitin, Sestrin 2 and Histone 3 in Sestrin 2 immunoprecipitates and their lysates were analysed. Representative images are shown (top). The mean values (bottom) of ubiquitinated proteins in Sestrin 2 immunoprecipitates are shown. Results are presented as a mean±SD of 4 repeated experiments. Student's T-test was used to analyse the data. **p<0.01

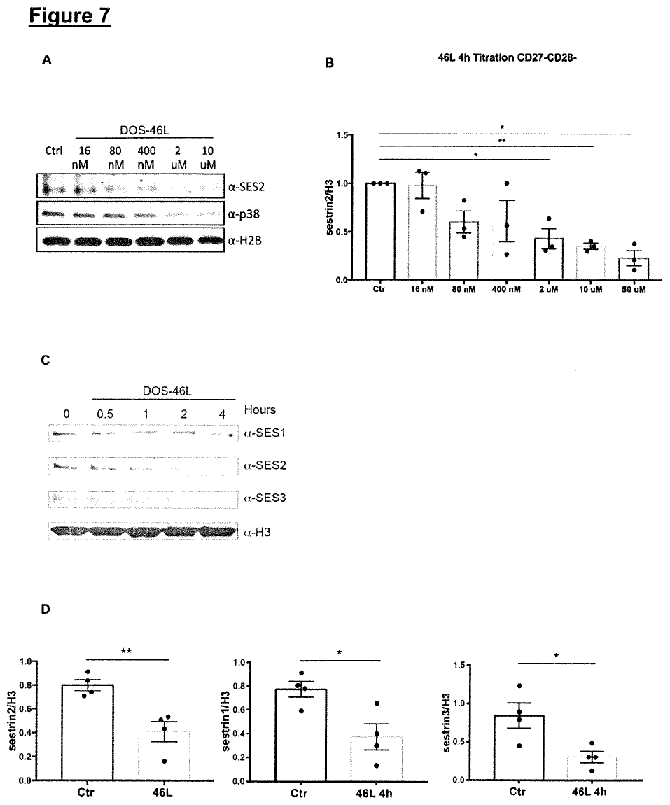

FIG. 7 illustrates titration of DOS46L. (A-B) CD4+ CD27− CD28− T cells were isolated from PBMCs from healthy donors and treated with increasing doses of DOS46L compound for 4 hours and harvested for immunoblot analysis. (A) Levels of Sestrin 2, p-p38 and H2B were evaluated by immunoblot. (B) Quantification of Sestrin 2 was analysed with Histone 3 as a loading control. Represented are the mean values±SEM of at least three independent experiments. Values are compared using One-Way ANOVA test followed by Tukey's post hoc test. (C-D) CD4+ CD27−CD28− T cells were treated with DOS46L compound (10 uM) for 0.5, 1, 2 and 4 hours and harvested for immunoblot analysis at indicated time points. (C) Levels of Sestrin 1, Sestrin 2, and Sestrin 3 were evaluated by immunoblot. Histone 3 was used as a loading control. (D) Quantification of Sestrin 1, Sestrin 2 and Sestrin 3, with Histone 3 as a loading control. Represented are the mean values±SEM of at least four independent experiments. Values are compared using One-Way ANOVA test followed by Tukey's post hoc test or Student's T-test. *p<0.05, **p<0.01

FIG. 8 shows binding of DOS46L to sMAC. (A) Biotinylated DOS46L was mixed with flag recombinant Sestrins, immunoprecipitated with streptavidin and immunoblotted with flag Sestrins. DOS46L directly binds to Sestrins. Representative images from three experiments are shown. (B—C) Recombinant sestrins do not bind to each others. (D) DOS46L binds to the entire sMAC complex in CD4 T cells. CD4+ T cells were isolated from PBMCs from healthy donors and treated with biotinylated DOS46L compound (10 uM) for 4 hours. Cells were harvested for immunoprecipitation analysis.

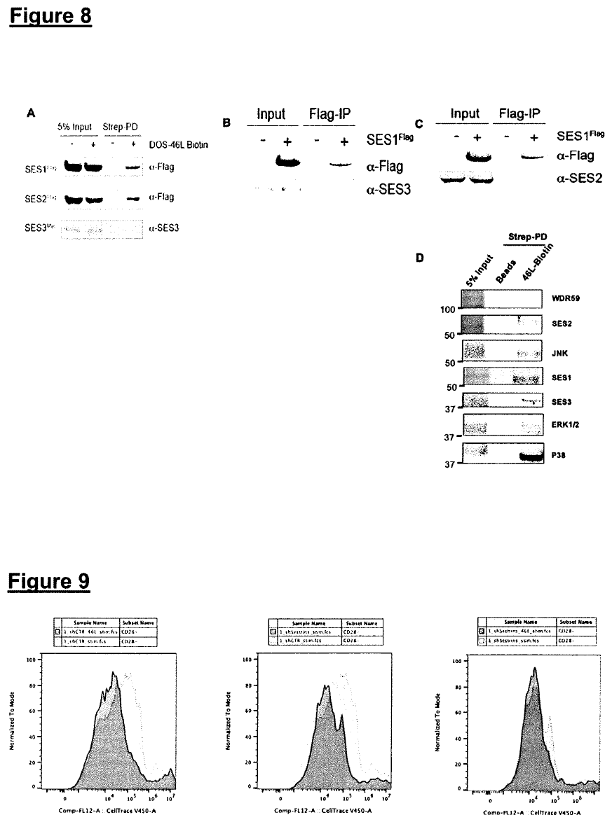

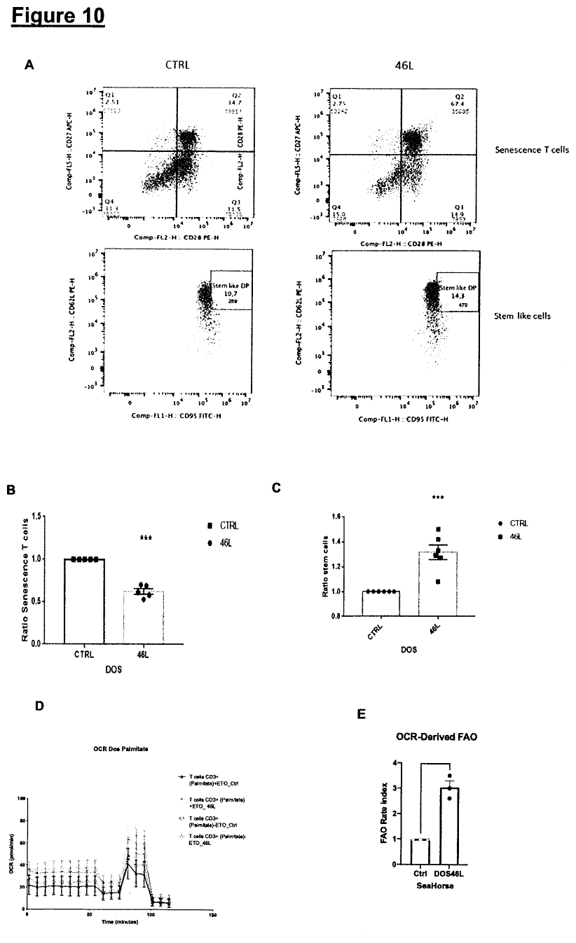

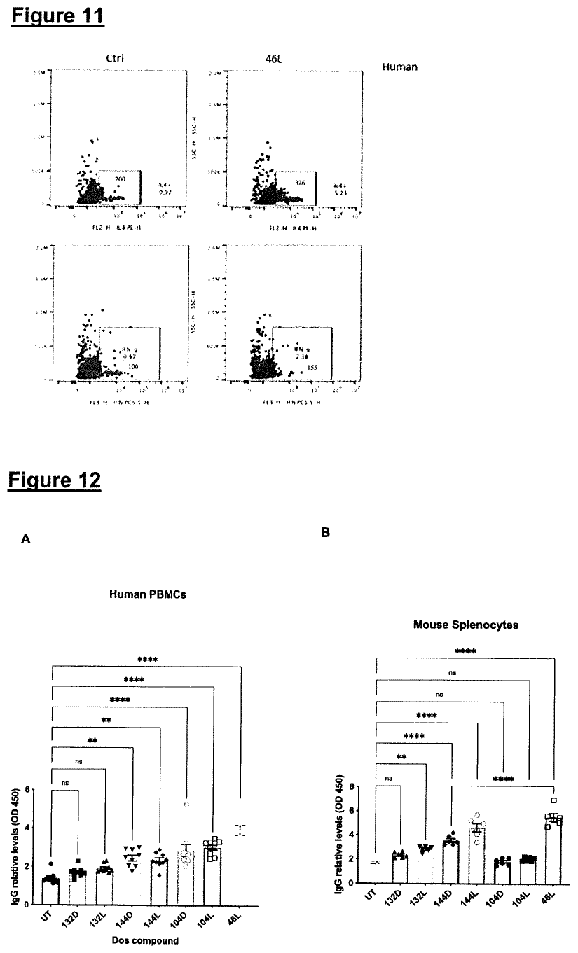

FIG. 9 illustrates cell proliferation in the presence of DOS, with or without sestrin. CD4+ CD27-CD28-T cells were purified from PBMCs from healthy donors, transduced with lentiviral vectors to eliminate sestrins, stained with CTV (Cell Trace Violet) and treated with DOS46L compound (10 uM) for 96 hours. Cell proliferation was assessed by FACS analysis. shCtrl T cells did express sestrins. Representative of nine experiments.

FIG. 10 illustrates immune-rejuvenation by DOS46L. (A) PBMCs were purified from PBMCs from healthy donors and treated with DOS46L compound (10 uM) for 12 days.

Senescent T cells and stem cells were analysed using FACS analysis. (B—C) Represented is the ratio to untreated sample. Each dot is an individual donor. (D) Oxygen Consumption Rate (OCR) of T cells treated with DOS46L for 24h were measured by Seahorse. Induction of Fatty Acid Oxidation (FAO) by DOS46L. (E) The FAO index was calculated by OCR difference between Ctrl and DOS46L treated T cells in the presence or in the absence of the FAO inhibitor etomoxir by Seahorse. Student's T-test was used to analyse the data. **p<0.01; ***p<0.001.

FIG. 11 shows cytokine production by DOS. PBMCs were purified from healthy donors and treated with DOS46L compound (10 uM) for 12 days, then analysed by FACS analysis. Cytokine analysis on senescent T cells after 12 days of DOS46L stimulation. The number of cytokine producing cells is underlined.

FIG. 12 shows in vitro IgG quantification by DOS. (A) PBMCs were isolated from whole blood and treated with various DOS compounds (10 uM). Levels of IgG were analysed by ELISA. (B) Murine splenocytes treated with DOS compounds (10 uM). Levels of IgG were analysed by ELISA. Anova with Tukey's post correction test was used to analysed the data. **p<0.01, ***p<0.001

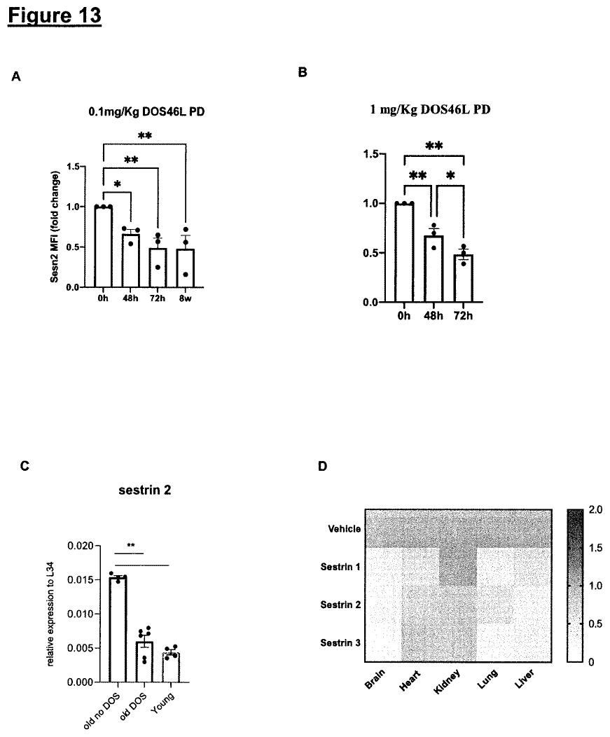

FIG. 13 illustrates the level of Sestrin in DOS-treated murine blood and tissues. (A-B) Old mice were inoculated subcutaneously with DOS46L compound (0.1 mg/kg or 1 mg/kg). Blood was withdrawn at 0, 48, 72 hours and 8 weeks after the inoculation. Pharmacodynamics of Sestrin 2 were analysed by FACS in CD4+ T cell population. (C) Long term mRNA silencing of sestrin expression in spleens from old mice 10 after single dose of DOS46L (0.1 mg/Kg). Untreated age-matched controls are shown. (D) 72 hr after DOS treatment, mice were sacrificed and old tissues were collected to analyse Sestrin levels in different organs by immunoblotting. **p<0.01, *p<0.05.

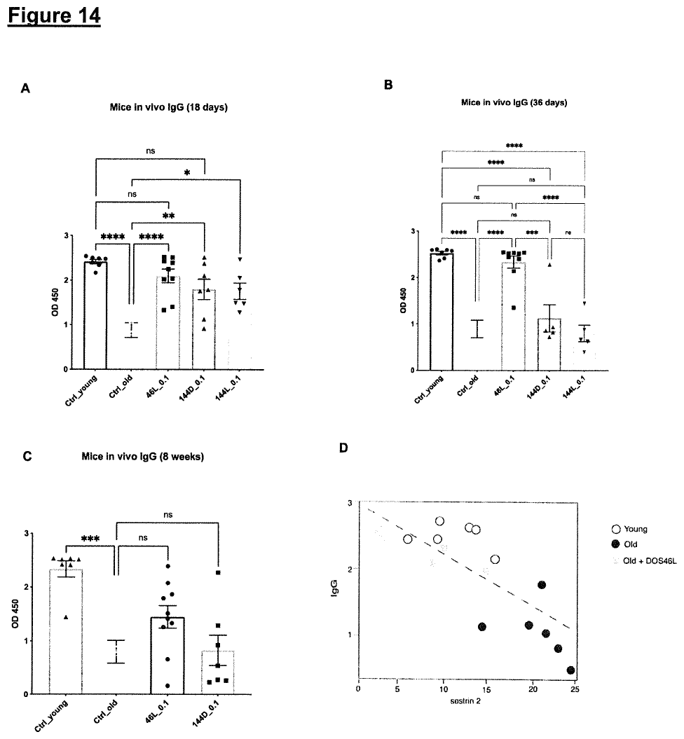

FIG. 14 shows long-term effects of DOS post-immunization. Old mice were inoculated subcutaneously with DOS compounds (0.1 mg/kg or 1 mg/kg). Blood was withdrawn 18 days (A), 36 days (B) and 8 weeks (C) post-immunization. (D) Correlation curve between IgG levels and Sestrin 2 in blood from mice after 18 days of treatment with DOS46. Student's T test was applied. ***p<0.001, **p<0.01, *p<0.05.

FIG. 15 illustrates the clinical effects of DOS with vaccination. Old mice were vaccinated in the presence or absence of DOS compounds and infected with H1N1 PR8/54 six months later. The mice were monitored for 14 days and their survival (A), clinical score (B) and weight (C) analysed. The disease severity is summarised in (D). Anova with Bonferroni post correction test. **p<0.001 ***p<0.001

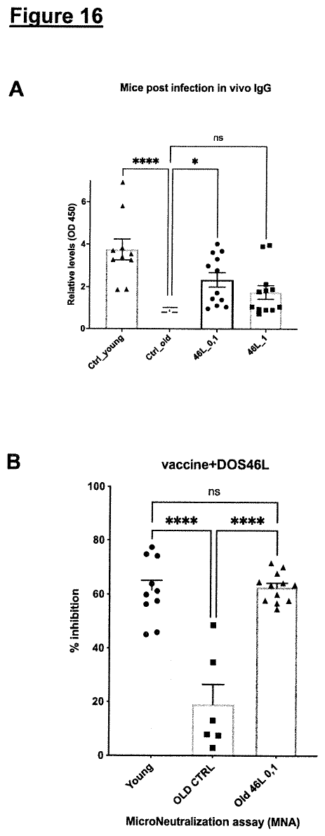

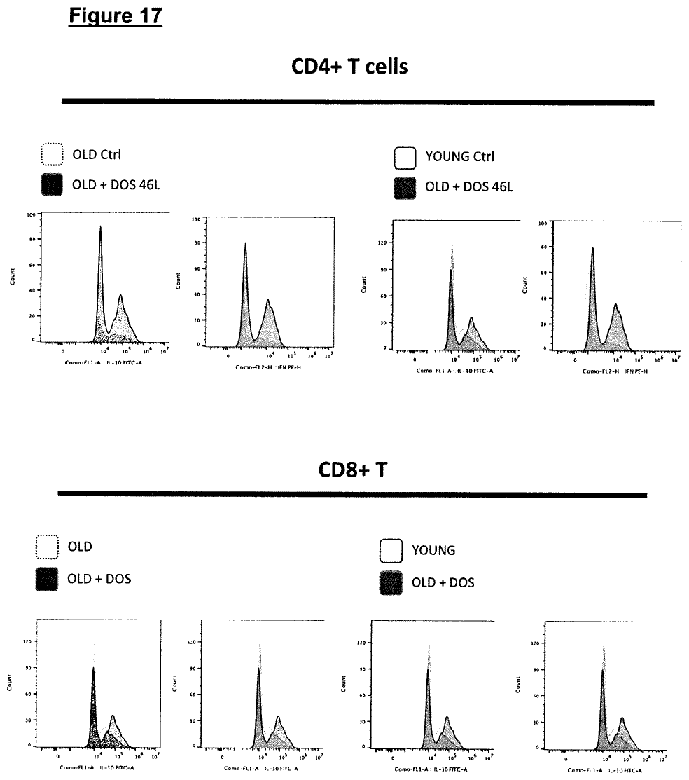

FIG. 16 shows the analysis of influenza specific IgG antibodies before and after infection and their neutralisation effect. (A) Presence of circulating influenza-specific IgG antibodies in the blood from young and old mice, vaccinated and infected with H1N1 PR8/54, was assessed by ELISA. (B) Neutralisation effect of serum from young and old mice vaccinated and treated with DOS46L (0.1 mg/Kg) was assessed 6 months later, followed by infection with H1N1 PR8/54 and microneutralization assays against the same virus. The graph shows the percentage of inhibition of viral growth. Each dot is an individual mouse throughout. Anova with Bonferroni post correction test was used ****p<0.0001; ns, not significant.

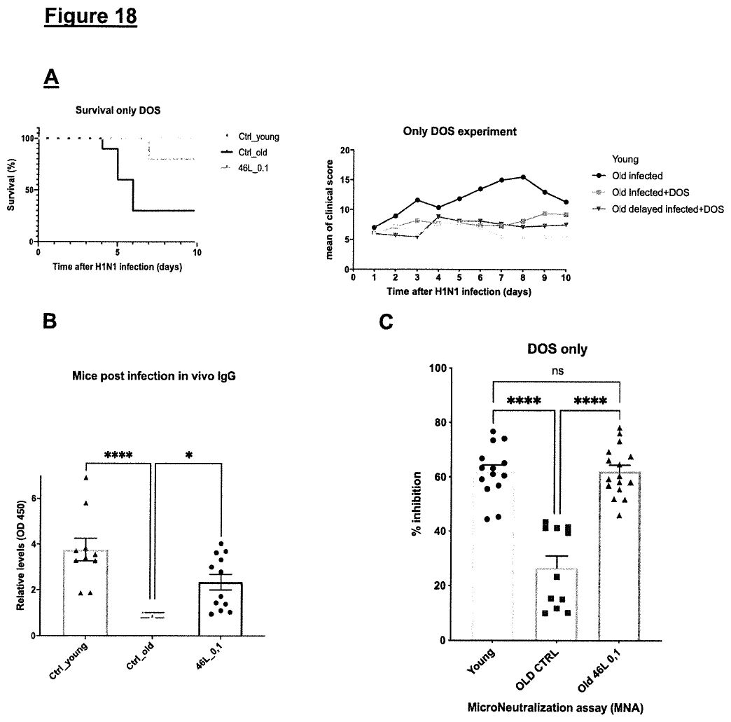

FIG. 17 shows in vivo immune activation by DOS in the lungs of infected mice. Cytokine production in the lung CD4+ and CD8+ T cells from old mice vaccinated with FLUAD, with or without DOS compounds (single dose), then infected with H1N1 PR8/54 six months later. Vaccinated young mice served as a control. Representative of 10 animals per group.

FIG. 18 illustrates survival data of DOS-only treated mice. (A) Old mice were treated with or without DOS, then infected either 72 hours or 10 weeks later (long term) with H1N1 PR8/54. The mice were monitored for 14 days and their survival, analysed. Young mice not treated with DOS are also shown. Note the prophylaxis action of DOS that protects mice from influenza infection (3.5*10\hat{ }5 viral particles) in the absence of vaccination, in both short and long term. The clinical score is shown on the right. Seven to ten mice per group are shown. Young mice served as a control. (B) IgG titres in old mice treated with DOS only for 10 days and infected with H1N1 PR8/54. Young and old controls are also shown. Each dot is an individual mouse. (C) Microneutralization assay performed on serum of mice long term infected since DOS treatment. Old and young controls are also shown. Note that the neutralising effects of DOS only are comparable to those of the vaccinated mice. Anova with Bonferroni post correction test. ****p<0.0001; ***p<0.001.

FIG. 19 shows viral clearance by DOS compounds from lungs of old mice. Lungs were taken at 6 days post infection. (A) Autoptic lung analysis and (B) viral clearance was measured by absence of the viral protein PA normalized to the housekeeping gene L34. qPCR was performed in cells from lungs of old mice, untreated or treated with DOS46L, two days before infection with H1N1. A Mann Whitney test was used *p<0.05.

FIG. 20 shows DOS effects on stem-like memory T cells in vivo. Time course of stem-like generation among CD95⁺ CD62L⁺ CD44⁻ CD4⁺ memory T cells was monitored by FACS analysis in the spleen of mice at day 10, 21, 30 and 8 weeks after injection with DOS-46L (0.1 mg). Results are representative of at least 3 mice per group.

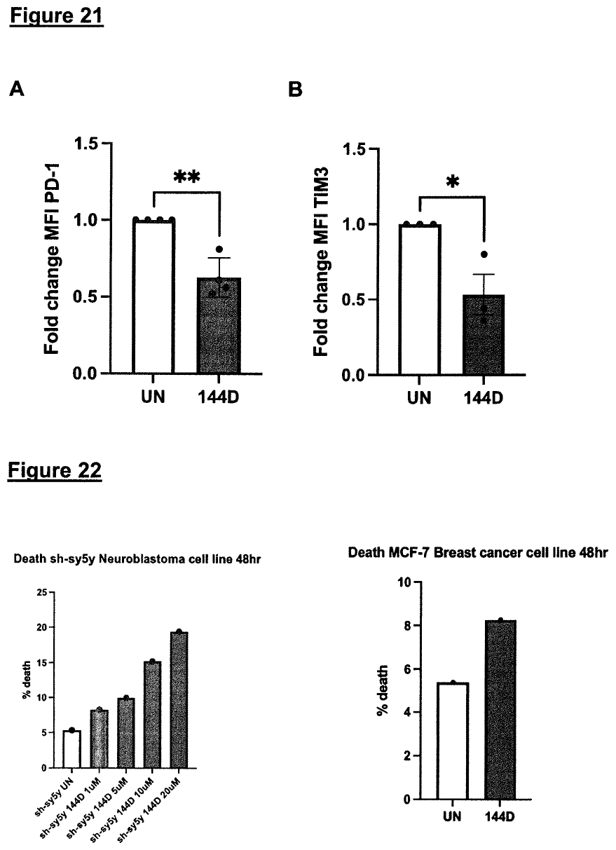

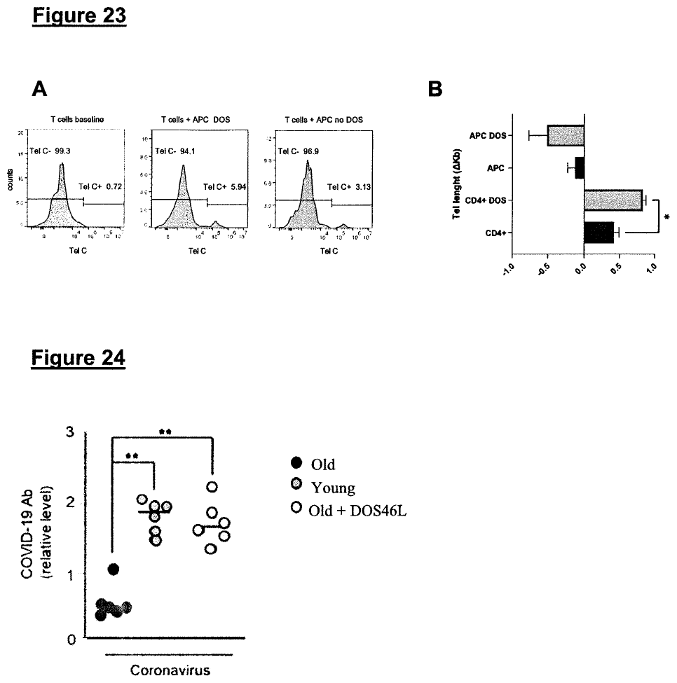

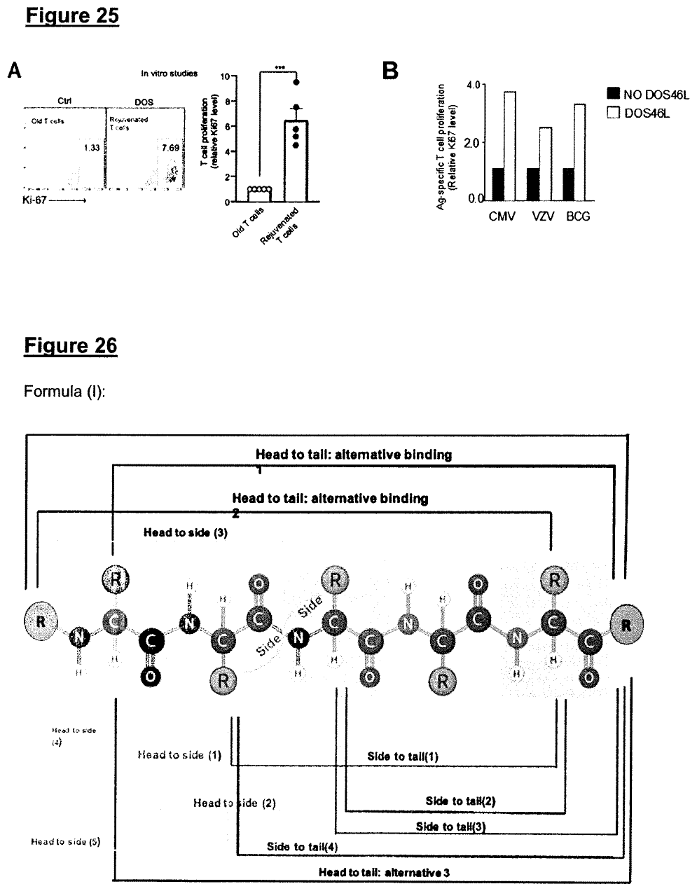

FIG. 21 shows checkpoint inhibition by DOS. PD1 and Tim 3 expression in primary human senescent CD27⁻ CD28⁻ CD4⁺ T cells activated with anti-CD3 and anti-CD28 for 12 days in the presence or absence of DOS (10 mM). Results are representative of 6 donors. The percentage of checkpoint inhibitor positive cells is shown. DOS inhibits checkpoint expression. UN depicts untreated controls. A student's t test was used. * p<0.05, **p<0.001.

FIG. 22 illustrates induction of cancer cell death by DOS compounds. (Left) Immortalized paediatric neuroblastoma cell line (sh-sy5y) was treated at different concentrations of DOS46L and DOS144D (from 1 uM to 20 uM) for 48 hours, then analysed by FACS with live-dead staining. (Right) Breast cancer cell line (MCF-7) was treated at the fixed concentration of DOS46L and DOS144D (20 uM) for 48 hours, then analysed by FACS with live-dead dye staining. UN depicts untreated controls. Results of triplicate cultures are shown.

FIG. 23 illustrates live transfer of telomeres from APC to T cells in the presence of DOS. (A, left): T cells showing baseline fluorescence in the channel of Tel C; (A, middle): T cells treated with DOS cultured with APC; (A, right): T cells co-cultured with APC, in the absence of DOS. DOS enhances telomere transfer by ˜2 fold. Representative of 3 experiments. (B): Telomere extension was quantified by qPCR. Synapse-driven telomere extension (Akb) in T cells exposed to APCs and treated with or without DOS46L. Results from 5 donors are shown. It is noted that the restoration in telomere length is similar to the extension observed in young T cells (Lanna (Group), 2020). Mann-Whitney was used. *p<0.05.

FIG. 24 shows stimulation of COVID-specific antibodies by DOS. PBMCs from elderly donors (75-95 years) were isolated from whole blood and treated with various DOS46L or DOS132L compounds (10 uM). Levels of IgG were analysed by ELISA. **p<0.01.

FIG. 25 shows induction of Ag-specific proliferation by DOS. Flow cytometry assay showing restored proliferation (Ki67*) in senescent human CD27⁻ CD28⁻ CD4⁺ T cells treated with DOS compounds for 3 days with surrogate CD3/IL2 activation (A) or APCs loaded with specific antigens (B). Note that DOS stimulate T cell proliferation independently of the antigen used. In (A), 5 donors, in (B) an experiment representative of 4. Student's T-test was used to analyse the data. ***p<0.001.

FIG. 26 shows Formula (I) and the different binding options to produce the cyclic polypeptides of the invention.

FIG. 27 shows docking of the cyclic polypeptides of the invention, in particular DOS46L, to sestrin 2.

FIG. 28 shows an alignment of the human sestrin 2 and human sestrin 1 amino acid sequence. * indicates a conserved amino acid; ·· indicates a chemical similar amino acid.

FIG. 29 shows an alignment of the human sestrin 2 and human sestrin 3 amino acid sequence. * indicates a conserved amino acid; ·· indicates a chemical similar amino acid.

FIG. 30 shows a comparison of the cyclic polypeptides (DOS compounds) derived from sestrin 1 and sestrin 2. The conserved amino acid residues are underlined. Cyclic polypeptides with conserved amino acid residues between sestrin 1 and sestrin 2 are named DOS alternative variants, i.e. “DOSₐₗₜ”. Cyclic polypeptides with no conserved amino acid residues between sestrin 1 and sestrin 2 are named selective DOS compounds, i.e. “DOSₛₑₗ”.

FIG. 31 shows a comparison of the cyclic polypeptides (DOS compounds) derived from sestrin 2 and sestrin 3. The conserved amino acid residues are underlined. Cyclic polypeptides with conserved amino acid residues between sestrin 2 and sestrin 3 are named DOS alternative variants, i.e. “DOSₐₗₜ”. Cyclic polypeptides with no conserved amino acid residues between sestrin 2 and sestrin 3 are named selective DOS compounds, i.e. “DOSₛₑₗ”. DOS_{con}***** indicates an alternative compound that is actually conserved between sestrin 2 and sestrin 3.

FIG. 32 shows the amino acid sequences of the DOSₛᵢₘ cyclic polypeptides for sestrin 1. DOSₛᵢₘ cyclic polypeptides are those where a blast search has identified a region of homology between sestrin 1 and 2 (chemical similarities) in a different position.

FIG. 33 shows the amino acid sequences of the DOSₛᵢₘ cyclic polypeptides for sestrin 3. DOSₛᵢₘ cyclic polypeptides are those where a blast search has identified a region of homology between sestrin 3 and 2 (chemical similarities) in a different position:

FIG. 34 shows the amino acid sequences of the DOS_{con} cyclic polypeptide between sestrin 2 and sestrin 1. DOS_{con} are those that overlap and/or have chemical similarities between any two sestrins.

FIG. 35 shows the amino acid sequences of the DOS_{con} cyclic polypeptide between sestrin 2 and sestrin 3. DOS_{con} are those that overlap and/or have chemical similarities between any two sestrins.

FIG. 36 shows the amino acid sequences of the DOS_{supercon} cyclic polypeptides. DOS_{supercon} are those that overlap and/or have chemical similarities among all three sestrins.

### EXAMPLES

The inventors have generated novel cyclic polypeptide inhibitors of sestrin-MAPK complexes (sMAC), named DOS (disruptors of sMAC), and have demonstrated that these polypeptide DOS compounds can inhibit sMAC in nanomolar amounts, by targeting sestrin for proteasomal degradation.

### Materials and Methods

In vitro screening inhibition of AMPK1 stimulation by Sestrin1 using the peptide library
1. Recombinant protein AMPK1 (5 ng) and DOS peptide (20 ng) were incubated for 30 minutes at RT;
2. Sestrin1/ATP/AMP/SAMsite (1 ug) were added and incubate for 1 hour at RT;
3. 20 μl ADP-Glo reagent were added and incubated for 40 min at RT;
4. Were then added 40 μl of kinase detection reagent and incubated for 30 min at RT
5. Results were acquired at the ELISA reader.

### Immune-Fluorescence for DOS Localisation in CD3+ Cells

1. Purified T cells were plated at the concentration 1*10⁶ cell/well in 24 well plate
2. Cells were treated with 10 μM of DOS-FITC for 4 hrs
3. Then collected and centrifuged at 500 g for 5 mins
4. Washed two times with ice-cold PBS
5. Cells were resuspended in 200 ul of PBS and plated in a pre-treated coverslip with poly-lysine
6. Cells were fixed with 4% of formaldehyde in ice for 20 mins
7. Washed three times with PBS
8. Blocked with PBS-BSA 5%
9. Cells were then incubated with primary antibody (anti-CD3 1:100) overnight at 4 C
10. Washed three times with PBS
11. Then incubated with secondary antibody (anti-mouse conjugated with AF-594; 1:250) for 1 hr at room temperature
12. Washed three times with PBS 13. Cells were permeabilized with Triton 0.25% in PBS for 2 mins
14. Followed incubation with antibody anti-KDEL (Abcam) and anti-sestrin 2 (Gentex) diluted 1:100 at 4 C over-night
15. Washed for three times with PBS
16. Secondary antibodies anti-mouse conjugated-647 or with anti-rabbit conjugated-594 diluted 1:250 was then incubated for 1 hr at room temperature
17. All washed for three times with PBS
18. Coverslips were mounted with Vectashield mounting media DAPI+
19. Images were acquired at the confocal microscope Zeiss

### Western Blotting Protocol

1. CD4+ cells were separated from PBMC using appropriate microbeads kit (Miltenyi)
2. Cells were resuspended in whole RPMI medium and plated in the 24-multiwell plate
3. Then activated for 4 hrs with DOS compounds
4. Cells were then harvested, washed with PBS and resuspended in lysis buffer (RIPA with phosphate inhibitors)
5. The cell-lysis mix was incubated for 20 mins on ice
6. Samples were centrifuged for 20 mins at the max speed
7. The supernatant was collected and Lemmli buffer+β-Mercaptoethanol was added
8. Samples were then boiled for 5 mins at 95° C.
9. Samples were loaded on the polyacrylamide gel and left to run in TRIS-Glycine-SDS running buffer
10. Once the run was completed the proteins were transferred from the gel to the nitrocellulose membrane by using the Trans-Blot machine (Biorad)
11. Membrane was washed with 0.1% Tween-TBS buffer and blocked in 5% non-fat milk for 1 hr
12. Followed incubation with the primary antibody o.n.
13. The day after, the membrane was washed with 0.1% Tween-TBS buffer for three times for at least 30 mins each
14. Secondary antibody was then added for 1h
15. The membrane was the washed with 0.1% Tween-TBS buffer for three times for at least 30 mins each
16. The signal was developed using ECL solution

### Immunoprecipitation of Sestrin2

1. Cell pellet was lysed with 100 μL of denaturing lysis buffer
2. Then mixed well by vortexing vigorously for 2-3 sec at maximum speed.
3. Samples were then boiled at 95° C. for 5 min.
4. Samples were then diluted 900 μl of non-denaturing lysis buffer and mixed gently.
5. Cell lysates were then passed through a needle attached to a 1 ml syringe for 5-10 times or were sonicated.
6. Incubated then on ice for 5 min
7. Samples were pre-cleaned by spinning at the max speed at 4° C. for 20 min.
8. Supernatants were collected in a new tube
9. 7 ul (5 μg) of SESN2 (abcam) primary anti-body was added directly to the supernatant and incubated on the rota o.n. at 4° C.
10. 200 ul of protein A/G beads were washed 4-5 times in IP buffer and blocked then o.n. in IP buffer with BSA and left on the rota at 4° C.
11. The day after, 50 ul of protein A/G beads were added to the sample and incubated for 3 hrs on the rota at 4° C.
12. Protein-beads conjugates were washed 5-10 times in IP buffer by spinning at 3000 g for 2-3 min
13. Then 1× Laemmli reducing agent was added and samples were boiled at 95° C. for 10 min
14. Followed Western Blot protocol
   In Vitro Pull Down from Protein Cell Extracts
1. 5×10⁶ cells cells were lysated in 200 ul of PE buffer (complemented with phosphatase and protease inhibitors) and incubated on ice for 30 minutes;
2. Cell extract was with 1800 μl of PD buffer;
3. The extract was pre-cleared by centrifugation at max speed for 30 mins;
4. 600 μl of the supernatant was then incubated o.n. with DOS-biotin at 4° C. (20 μM concentration);
5. Streptavidin-agarose beads (1000 ul) were washed with PD buffer and blocked o.n. with 5% BSA;
6. Beads were washed again and incubated with the cell extract for 1 hr at RT
7. Followed washing of beads with PD buffer
8. Proteins were eluted with 50 ul of Laemmli 1×

### Lentivirus Production in HEK293 Cells

1. Day 0: HEK293 cells were plated in the cell culture flasks t75, at certain concentration to have 70% confluence the day after. (for each lentivirus were plated 2 flasks t 75 of HEK293 cells).
2. Day 1: HEK293 cells were transfected by using Lipofectamine LTX reagent with PLUS reagent (Invitrogen);
3. Specifically, before transfection, the media was removed and was added 5 mL Opti-MEM reduced serum medium (Gibco) to each flask;
4. The plasmid DNA-PLUS lipid complex was obtained by combining 4 μg of DNA with 10 ul Lipofectamine 2000 and PLUS reagent in a ratio 1:1. The mix was incubated for 10 mins at RT.
   For the production of lentiviral particles, cells were co-transfect with the following three plasmids: pCMV-VSV-G envelope vector (Cell Biolabs);
   p.891 packaging vector; p-SIREN-scramble/p-SIREN-shsesn1/p-SIREN-shsesn2/p-SIREN-shsesn3.
5. The DNA-lipid complex was then added to cells dropwise;
6. 5 hours after transfection, the media was carefully aspirated and replaced with 15 mL of fresh DMEM complete;
7. Cells were incubated at 37° C., 5% CO2 for 48 hrs.
8. Day 3: Media containing lentivirus was collected at 48 hrs post transfection and cells were replaced with fresh media.
9. Day4: Media containing lentivirus was also harvested at 72 hours post transfection.

### Lentivirus Concentration

1. Lentiviral particles were concentrated using Lenti-Pac Lentivirus Concentration Solution (GeneCopoeia).
2. The media containing lentivirus particles was centrifuged at 2000 g for 10 mins at 4° C.;
3. And then mixed with Lenti-Pac Concentration Solution at ratio of 5:1;
4. Incubated at 0-4° C. o.n. and centrifuged at 3500 g for 25 mins at 4° C.;
5. Supernatant was carefully discarded
6. The viral pellet was resuspend with 100 μL of PBS
7. Virus aliquots were stored at −80° C.

### CTV Staining

1. Transduced cells were collected
2. Cell suspension was centrifugated and the cell pellet resuspended in warm PBS with 5 μM of CTV
3. Followed an incubation of 10 min at 37° C.
4. Cold PBS was added and cells were centrifugated
5. Cells were then resuspended in medium and counted
6. An aliquot of these cells were checked by FACS
7. Cells were then stimulated with DOS in 200 μL and left for 12 days in culture
   RNA Extraction, Retro-Transcription and qPCR
1. CD4+ cells were purified from mouse spleen using appropriate microbeads (Miltenyi)
2. Total RNA from CD4+ cells was extracted using the “PureLink RNA Mini Kit” (Invitrogen).
3. RNA concentration was acquired by NANO-DROP spectrophotometer (Thermo-Scientific).
4. 1 μg of total RNA was retro-transcribed to cDNA following the protocol advised from the kit “qScript cDNA synthesis kit” (Quanta Biosciences).
5. qPCR reaction was performed with 100 μg of cDNA per sample using the kit SensiFAST Hi ROX kit (Bioline).
6. For investigation of human sestrins the following Taqman probes were used:
   Sestrin 1 Hs00902782_m1,
   Sestrin 2 Hs00230241_m1,
   Sestrin 3 Hs00914870_m1,
   RPLPO Hs00420895_gH (used as house-keeping control)
For investigation of mouse sestrin 2 the following primers were used:

Fw TCTCGGCACTTTGAGGACAC
Rw AACCATGGTCTTCCCAGCAG

Standard primers for RPL34 were used as house-keeping control
For investigation of the PA viral protein of H1N1 virus were used the following primers:

Fw CGGTCCAAATTCCTGCTGA
Rw CATTGGGTTCCTTCCATCCA

7. The qPCR reaction was conducted by using “StepOne Plus System” machine (Applied Biosystems)

### Seahorse Assay for Cell Metabolism

1. CD4+ cells were purified from PBMC using appropriate microbeads (Miltenyi)
2. Purified CD4+ cells were activated with CD3/CD28 Dynabeads (Gibco) and treated over-night with DOS46L at 10 μM.
3. Day after, the conventional growth media was removed from the cells and was added the “substrate-limited growth media” composed of basic DMEM plus 0.5 mM glucose, 1 mM glutamine and 1% FBS.
4. Cells were then incubated with this media for 4 hrs in incubator.
5. Cells were then counted and plated at the concentration of 2*10⁵ cells/well in the XF96 well plate pre-treated with poly-lysine.
6. Cells were then supplemented with the “assay media” composed of XF DMEM with 2 mM of glucose and 0.5 mM L-carnitine.
7. Just before to run the assay, cells were treated with 1×palmitate-BSA or with 1×BSA.
8. During the assay, cells were treated at different time points with Etomoxir (4 uM), Oligomycin (1.5 μM), FCCP (1 μM) and Rotenone/Antimycin A (0.5 μM).
9. Seahorse machine took reads of OCR values at different time points for the time span of 120 mins.
   PBMC In-Vitro Treatment with DOS
1. PBMCs were de-frosted and resuspended in 5 mL of medium
2. Left in incubator o.n.
3. CD4+ CD27-CD28-were purified using appropriate microbeads (Miltenyi)
4. Cells were then stimulated o.n. with CD3 CD28 Dynabeads (20 uL for 10\hat{ }6 cells) in 96 well plate flat in presence or not of DOS compounds
5. Cells were left in incubator for 12 days and then they were analysed.

### Determine IgG Production Following Immunisation

1. Analysis of plasma IgGs specific for the virus H1N1 was performed at 18 and 36 days after immunisation by ELISA
2. The antigen (FLUAD) was coated in a 96 well plate; 50 μl per well, dilution 1:40 (12 ml of PBS+300 μl FLUAD)
3. Then washed in PBS 0.5% Tween-20
4. And blocked with 4% milk in PBS 0.5% Tween-20, 2 hrs at RT
5. Mice plasma was then added to the blocked plate at the dilution 1:200 in 0.1% milk PBS 0.5% Tween-20
6. Plate was incubated o.n. at 4° C.
7. Washed once with PBS 0.5% Tween-20
8. The anti-mouse IgG1 biotin conjugated antibody diluted 1:250 in 0.1% milk PBS 0.5% Tween-20 was then added and incubated for 2 hrs at RT
9. Plate was washed twice with PBS 0.5% Tween-20
10. Streptavidin-HRP conjugated antibody, diluted 1:3000 in PBS 0.5% tween-20 was added and left for 1 hr at RT
11. Followed three washes with PBS 0.5% Tween-20
12. TMB substrate was added to the wells and left for 15-30 min until the colour appeared
13. The reaction was stopped with the stop solution (ThermoFisher)
14. The absorbance was detected at 450 nm by ELISA reader.

### Telomere Live Transfer

1. CD4+ cells were purified from PBMC using appropriate microbeads (Miltenyi)
2. CD4+ cells and CD4-cells were collected
3. CD4+ cells were subsequently used to purify CD27− and CD28− cells through the Miltenyi purification kits.
4. 1*10⁷ CD4-cells were plated in a spot, at the centre of the well (6 well plate), treated with DOS 46 L 10μM and left to attach for 2.30 hrs.
5. In parallel, also 5*10⁵ CD27−/CD28− cells were treated with DOS 46L 10 μM for 2.30 hrs.
6. CD4-cells were then stained with TelC-Cy5 probe (PNA bio Link) diluted 1:50 in PBS for 5 mins
7. In each well containing the stained CD4-cells were added glass beads; the plate was gently moved for 2 minutes to make pores in the cell membrane
8. Cells were washed with PBS and centrifuged
9. The pellet was resuspended and CD4− cells were counted
10. CD4+ cells were mixed with CD4− in a ration 1:3
11. Then, combined CD4+ and CD4-cells were stimulated with a mix 1:1 of CMV and EBV
12. Cells were incubated overnight
13. Day after, cells were stained for surface proteins and analysed at the FlowCytometer.

### Micro-Neutralization Assay

1. Mouse sera were diluted 1:200 and incubated with H1N1 virus diluted 1:10000 for 1 hr at 37° C.
2. MDCK cells were then added at the concentration of 15.000 cells per well (96 well plate) and incubated over-night at 37° C. with 5% of CO₂.
3. The day after, cells were fixed with cold Acetone 80% in PBS
4. The primary antibody anti-influenza A NP, diluted 1:250, was added to the wells and incubated for 1 h at RT
5. Plate was washed twice with PBS-Tween 0.3%
6. A secondary antibody (goat anti-mouse IgG HRP labelled) diluted 1:2500 was added and the plate was incubated for 1 hr at RT.
7. Plate was washed twice with PBS-Tween 0.3%
8. The TMB substrate was added to each well and left until the colour change.
9. The reaction was stopped with the stopping solution (Thermos Fisher) and the O.D was acquired at the ELISA reader at 750 nm.

### In-Vitro Kinase Assay

1. Sestrin or AMPK Immunoprecipitates were washed twice in lysis buffer and twice in kinase buffer (all from Cell Signaling).
2. Immunoprecipitates were left untreated or treated with 10 uM of cyclic DOS compounds, in presence or not of 200 uM ATP (Cell Signaling) for 30 min at 30° C.
3. The sample were resuspended with PBS 4. An ELISA plate was coated with the kinase reaction product at 4 C o.n.
5. The plate was then blocked with 4% milk in PBS-Tween 0.5% for 1 hr.
6. MAPK activity was assessed by using antibody anti-phospho p38 (D3F9, Cell-Signalling), or with anti-phospho-SAPK/JNK (81E11) (Cell Signalling) and phospo-p44/42 MAPK (ERK 1/2) (D13.14.4E) (Cell Signalling)-. All antibodies were diluted 1:100 and incubated for 2 hrs at RT.
7. The plate was washed twice with PBS-Tween 0.5%
8. The secondary anti-rabbit IgG-HRP conjugated antibody (Millipore) diluted 1:2000 was added to each well and left for 1 hr at RT.
9. Plate was washed three times with PBS-Tween 0.5%
10. TMB substrate was added until colour appeared; the reaction was stopped with stop solution (ThermoFisher)
11. The absorbance was read at ELISA reader.
12. The in-vitro kinase assays data were shown as proportional to fold increase at 450 nm absorbance emission of triplicate wells±s.e.m, normalized to the total amounts of co-immunoprecipitated MAPKs.

### Mice Survival

1. Mice (strain CS7B) were injected with Fluad vaccine (GSK) at 1:20 combined with the DOS (46L, 144D and 144L) at two different doses 0.1 mg/kg and 1 mg/kg. Group number=8
2. Mice were divided in 5 groups, 8 animals for group. Groups were the following ones:
   GROUP 1: Old Mice (18 months old) vaccinated with Fluad without DOS;
   GROUP 2: Old Mice (18 months old) vaccinated with Fluad+DOS 46L at the two doses;
   GROUP 3: Old Mice (18 months old) vaccinated with Fluad+DOS 144D;
   GROUP 4: Old Mice (18 months old) vaccinated with Fluad+DOS 144L;
   GROUP 5: Young Mice (8 months old) vaccinated with Fluad without DOS
3. 4 months post vaccination, mice were infected with H1N1 flu virus (viral pfu 6*105)
4. The mice were monitored for 15 days: body weight, disease progression, clinical score and survival data were acquired.
5. After 15 days the mice were sacrificed and tissue were collected (blood, lung, spleen and thymus).

### Kinetics Dos

1. Mice (young and old) were injected sub-cutaneously with DOS 46L (0.1 mg or 1 mg) then bleed from the cheek at 2 hrs, 4 hrs, 24 hrs, 48 hrs
2. PBMCs were purified
3. Cells were stained for T cell surface proteins for 30 mins
4. Washed with PBS
5. Cells were fixed with PBS-Fixation buffer (1:1) and incubated for 10 min at 37 C
6. Washed with PBS and centrifuged
7. Cold Perm Buffer (BD) was then added to the cells and incubated for 20 mins in dark at 4 C
8. Washed with PBS
9. Cells were stained with the antibody anti-sestrin 2 (1:100) (GenTex) for 20 mins in ice
10. Washed in PBS
11. Cells were resuspend in PBS and analysed at the FACS machine
   Mice Treated Only with DOS and Infected with H1N1 Virus
1. Mice (strain CS7B) were divided in three groups:
   Group 1:10 old mice (18 months old) injected subcutaneously with DOS 46L at 0.1 mg/kg
   Group 2:10 old mice (18 months old) not injected with DOS
   Group 3:3 young mice (8 months old) not injected with DOS
2. 48 hrs post DOS administration all mice from all groups were infected with the virus H1N1 intra-nasal way at the concentration 3.5*105 PFU per mouse.
3. Clinical score was recorded for each mouse from day 1 to day 10 from the infection. The clinical score includes the following parameters evaluation:
   Body weight
   Behaviour
   Fur condition
   Chest breath
   Eye condition
4. Mice were sacrificed when they reached a clinical score of 13 (grade of severity accepted for ethic commission). Tissue were collected from each mouse (blood, lung, spleen and thymus).

### Telomere Length Quantification Assay

1. CD4− and CD4+ cells were purified from PBMC using appropriate microbead kit (Miltenyi)
2. CD4+ cells were then used to purify CD27− and CD28− cells by using Miltenyi purification kits.
3. A pellet of CD4− and CD4+/CD27−/CD28− cells were frozen.
4. 4*10⁶ CD4− were mixed with 1*10⁶ CD4+/CD27−/CD28− cells in presence or not of DOS 46L 10 μM.
14. The mixed cells were stimulated o.n. with a mix 1:1 of CMV and EBV
15. The day after, CD4− cells were detached from conjugates with CD4+/CD27−/CD28− cells with a syringe by using cold PBS
5. CD4− were then purified from CD4+ cells by using Miltenyi purification kits.
16. CD4− and CD4+ cell pellets post conjugate were then frozen.
17. Genomic DNA from frozen cell pellets was extracted by using the PureLink Genomic DNA Mini Kit (Invitrogen)
18. qPCR reaction was performed according to the protocol suggested by the “Absolute human telomere length quantification qPCR Assay kit” (ScienCell-Research Laboratories)
19. Telomere length was calculated from the ACT, according to the formula suggested by the kit mentioned above.

### Design of DOS Compounds

The inventors designed the DOS compounds on the protein Sestrin 2. However, to extend the DOS compounds to those that could potentially be active for Sestrin 1 and Sestrin 3, the inventors aligned the DOS compounds designed for Sestrin 2, against the protein sequences of Sestrin 1 and 3, as shown in FIGS. 28 and 29.

In doing so, the inventors identified DOS compounds that specifically bind to Sestrin 1 or Sestrin 3, but not to Sestrin 2, and so named these selective DOS compounds. For these compounds and for all the compounds that partially matched the Sestrin 2 sequence, the inventors predicted, according to a specific formula, the possible chemical binding that could naturally or synthetically happen between the reactive chemical groups present in the amino acids that make up the DOS sequence.

To report all of the possible secondary cyclic structure formations that could lead to the formation of functional compounds, the inventors used a specific code for each type of binding. Then for each code, they linked the letter of the amino acid involved in the binding. Specifically, if an amino acid has more than one specific reactive group, they assigned a number for each group to the letter of the amino acid. For example, Arginine presents three NH groups available and the inventors named them R2, R3 and R4. They used the code R′ to refer to the additional NH group available when the arginine is located at the N terminal of the amino acidic sequence.

### DOS Synthesis

To synthesise the cyclic peptide DOS compounds, conventional protection and activation chemistry was used, such that the amino functionality of a first amino acid was protected with a removable amino protecting group (e.g. benzoyloxy carbonyl (Cbz), tert-butoxycarbonyl (t-Boc), and 9-floure n-methoxycarbonyl (FMOC)), and the carboxyl functionality of a second amino acid is protected with a removable carboxyl protecting group. The carboxyl group may be protected by forming an acid or base labile ester such as a methyl, ethyl, benzyl, or trimethylsilyl ester. After protection, the first and second amino acids are reacted in a solution comprised exclusively of the respective L/D-amino acids, i.e. comprised of L/D-Arg, L/D-Gly and L/D-Asp. Following the coupling reaction, selective deprotection of the amino group of the first amino acid is accomplished by acid hydrolysis under conditions that do not remove the carboxyl protecting group of the second amino acid. The procedure is repeated with an additional amino protected. Solid phase synthesis such as well-known Merrifield, FMOC and MeDBz resins, may be used for synthesizing the DOS peptides of the invention. A linear peptide is first derived from natural sestrins, followed by cyclization.

### Example 1—Discovery of DOS Compounds

To develop first-in class inhibitors of sestrins, the inventors designed minimal in vitro assays with bacterially-purified proteins (sestrin 1 and sestrin 2). Sestrins target AMPK, the heterotrimeric alpha-beta-gamma kinase, which coordinates sestrin-dependent MAPK activation within the sMAC. Following activation of AMPK, detected by ATP mediated kinase reaction, 156 different sequences were tested by in vitro ELISA assay, and the results are shown in FIG. 2.

The pentameric sequences were rationally designed on a region between amino acid redsidues 1-160 of sestrin 2 (SEQ ID No: 4), because this whole region was shown to be essential for sestrin-AMPK interaction. Additionally, five amino acids is considered to be the limit length to generate cell penetrating peptides with inhibitory functions. This approach identified a number of potential inhibitory sestrin fragments (ISFs), 74 for sestrin 2 and 50 for sestrin 1, which were able to abrogate AMPK activity by 40-100% in the assay (see FIG. 2). The remaining sequences were unable to inhibit AMPK, and therefore, these were excluded from further analysis.

However, the inventors discovered that incubation of T cells with the ISFs failed to result in inhibition of sestrin or AMPK phosphorylation, suggesting limited cell penetrability and/or stability. Therefore, to overcome these challenges, the inventors generated head to tail cyclic peptidomimetics (both L and D-variants) for the ISFs identified in the in vitro screening. For reasons explained below, the inventors renamed these cyclic peptidomimetics disruptors of sMAC (DOS) compounds.

### Example 2-Ability of DOS Compounds to Inactivate sMAC

Among all of the DOS compounds tested, the inventors identified DOS46L and DOS144D as the most effective compounds at sMAC inactivation. For example, DOS46L silenced the sMAC pathway by 90%. Therefore, by way of example, the inventors focused on DOS46L (SEQ ID No: 22) and DOS144D (SEQ ID No: 77) for the majority of the following studies.

The inventors next carried out titration studies of DOS compounds, DOS46L and DOS144D, on AMPK1/Sestrin2 activity. As illustrated in FIG. 3, the titration studies demonstrated that DOS compounds can suppress sestrin-driven AMPK activation in nanomolar amounts with recombinant proteins in vitro (DOS46L, IC50 0.096 mM; DOS144D, 0.844 mM), demonstrating potency of the newly-generated DOS compounds.

The inventors next tested the ability of DOS compounds to penetrate T cells. As shown in FIG. 4, DOS compounds (DOS46L) were able to penetrate T cells and, in particular, the senescent CD3+ CD4+ CD27− CD28− population. Similar results were obtained with CD8+ T cells and major antigen presenting cells (APCs), including dendritic cells, monocytes, macrophages and B cells. When DOS compounds were added to APCs, they triggered spontaneously the release of telomere vesicles that could be used for rejuvenation.

When tested in total CD4+ T cells at 10 μM concentration, DOS compounds inhibited the entire sMAC signalling, as illustrated in FIG. 5. This was shown by downregulation of sestrin 1, 2 and 3 expression, and AMPK and MAPK phosphorylation. As such, this demonstrates that active sestrin inhibition leads to the disruption of sestrin-dependent sMAC activation, resulting in the downregulation of MAPK signalling.

As shown in FIG. 5, some DOS compounds have the ability to inhibit all three sestrins (e.g. DOS46L and DOS144D) and therefore, can be defined as pan-inhibitors. Others inhibit functionally only one or two sestrins (e.g. DOS104D and DOS117L) and therefore, can be defined as selective inhibitors. Accordingly, this demonstrates the possibility of selecting either a pan or selective inhibitor to inactivate sMAC, depending on the condition to be treated. For instance, a certain therapeutic approach may benefit from pan-inhibition of sestrins (e.g. by DOS46L), such as vaccination against bacterial, viral or parasite infections, whereas other conditions, such as epilepsy, are controlled by sestrin 3 and may benefit from a selective sestrin 3 inhibitor, such as DOS117L.

Using ELISA coupled to sestrin 2 immunoprecipitation, the inventors next confirmed that the addition of DOS compounds (DOS46L) directly to the sestrin immunoprecipitates in vitro, also disrupted sestrin-AMPK interaction that would suppress global MAPK activation (ERK, p38 and JNK) within the sMAC complex (FIG. 6). For example, as illustrated in FIG. 6A, the binding of AMPK to Sestrin 2 in CD4+ T cells decreased following 1 or 2 hours of treatment with DOS46L.

Referring to FIG. 7, titration studies in senescent CD4+ T cells exposed to DOS46L, demonstrated that the DOS compound suppressed sMAC activation within 4 hours of treatment, starting at nanomolar amounts (16 nM). This is similar to earlier observations made with recombinant proteins, confirming potency of the newly-generated inhibitor in real cells.

The treatment of CD4+ T cells with the proteasome inhibitor, MG132, and DOS46L, suggested that inhibition of sestrin, as well as AMPK, is due to proteasomal-mediated degradation. This is because the effect is vanished in cells pre-treated with the proteasome inhibitor, as shown in FIG. 6B. The inventors also immunoprecipitated an increased amount of ubiquitinated sestrin 2 in T cells that had been exposed to DOS46L (FIG. 6C). Therefore, these data demonstrate that DOS compounds are first in class inhibitors of sMAC, which target the complex at the point of sestrin, thereby inducing sMAC degradation through ubiquitination.

Referring to FIG. 8, an immunoprecipitation with a biotinylated variant of DOS46L and immunoblot to flagged sestrins showed that DOS directly binds to sestrin 1 and sestrin 2 (FIG. 8A). No sestrin was found in control precipitates with free biotin, demonstrating direct DOS46L-sestrin binding in vitro. By contrast, the sestrins did not bind to each other suggesting that they need to be within the sMAC to be found together in a complex (FIG. 8B-C). In real cells, the biotinylated DOS46L was found in the sMAC (sestrin 1, 2, and 3, as well AMPK and MAPKs), but not with elements of the GATOR complex (WDR59 and mTOR; FIG. 8D). The selective interaction was also studied using the cutting-edge AlphaFold algorithm for molecular docking predictions based on the deposited sestrin 2 crystal structure. These studies revealed that the compound likely binds in the domain A of sestrin 2 at the interface with domain B and C and, importantly, far away from the GATOR/mTORC1 regulatory site (domain C; FIG. 27). These data show that DOS are first in class selective sMAC inhibitors.

The specificity of DOS compounds was further tested in ‘sestrin-null’ T cells generated by shRNA delivery. DOS compounds restored senescent CD4+ T cell proliferation in shCtrl (Control) but not in sestrin-null T cells (FIG. 9); this was confirmed in all nine donors tested. This finding supports the fact that DOS compounds modulate T cell function via the sestrins.

### Example 3—Ability of DOS Compounds to Rejuvenate the Immune System

Immunophenotyping of PBMCs from healthy donors treated with DOS for 12 days showed reduction of senescent CD4+ T cells by 50%, and a concomitant increase of stem-like memory T cells and naïve T cells that are characteristics of immune rejuvenation (FIG. 10A-B). The rejuvenated capacity is also demonstrated by enhanced oxidative metabolism and fat burning (FAO) in DOS-treated T cells (Seahorse experiments) (FIG. 10 C-D).

The same was true in senescent CD8+ T cells. In addition, analysis of cytokines in stimulated CD4+ T cells revealed an increased production of IFN gamma, TNF alpha and IL-4 upon DOS treatment, which are all well-established factors that promote T cell-B cell collaboration and antibody production (FIG. 11) (Bot et al 2000).

In fact, addition of DOS to primary human peripheral mononuclear cells (PBMCs) isolated from older donors (age 70-85) was sufficient to restore influenza-specific IgG antibody production in vitro, back to the levels observed in young donor cells (age 20-40) not treated with DOS (FIG. 12A). This effect was also observed in murine splenocyte cultures, which reached up to three fold influenza-specific IgG antibody production (FIG. 12B). Strikingly, the inventors obtained similar results when using heat-inactivated SARS-COV2 (FIG. 24). As such, these data demonstrate that DOS compounds can be used as an effective vaccine booster.

DOS activity in vivo was tested at two different doses. Inhibition of T cell sestrin was robust in the blood of mice injected with DOS compound and was evident after 48 hours and peaked at 72 hours (up to 80% sestrin inhibition), and persisted, stably, up to 8 weeks (FIG. 13A). The long term sestrin downregulation was likely due to transcriptional silencing because samples from DOS treated mice demonstrated reduced mRNA (Lanna et al., 2017) sestrin expression starting from day ten of DOS administration compared to untreated age matched animals and similar to the expression levels present in the young mice. Therefore, DOS inhibit sMAC by a three tier mechanism; first they induce disruption of AMPK-sestrin regulatory binding (e.g. within 2 hours); second they target the dissociated proteins for ubiquitin mediated proteasome degradation (e.g. within 4h); finally, they transcriptionally silence sestrin mRNA expression (e.g. starting from day 10) resulting in permanent rejuvenation upon single dose administration. Young and old mice showed similar trends, demonstrating the ability of DOS to inhibit sestrins at different ages. The fact that the compound is effective in old mice at a very low dosage (0.1 mg/Kg) suggests enhanced specificity for old T cells that only possess sMAC, but not mTOR. Furthermore, immunoblotting on harvested organs demonstrated sestrin inhibition not only in circulating blood cells, but also in the brain, heart, liver, kidney and lung (FIG. 13C). This illustrates that DOS may therefore be employed in a variety of conditions characterized by sestrin/sMAC activation.

The long lasting pharmacodynamic effects prompted the inventors to test whether a single injection of DOS would be sufficient to restore vaccine responsiveness in old mice (median age 16 months). Strikingly, a single dose administration of DOS46L (0.1 mg/Kg) along with the vaccine, completely restored influenza specific antibody titres at 2, 4 and 8 weeks after vaccination, to levels observed in young mice (median age 3 months) vaccinated in the absence of DOS (FIG. 14A-C). The in vivo IgG stimulation effect is likely sestrin dependent as it correlates strongly with DOS-induced T cell sestrin down-regulation in blood (FIG. 14D). Similar results were obtained with another sMAC inhibitor, DOS144D. These data demonstrate restored vaccine responsiveness during aging by first in class sMAC inhibitors in vivo.

The long-lasting protection was tested by lethal inoculation of 6×10⁵ PFU live H1N1 PR8/54 viral particles six months after vaccination with DOS and monitoring for 14 days. FIG. 15 shows 100% survival in DOS treated mice upon infection (n=8), identical to young vaccinated mice (n=8). In contrast, the old cohort vaccinated in the absence of DOS showed just 15% survival (n=7). The survival data are similar for DOS46L and DOS144D, yet DOS46L is more effective at a very low dose (0.1. mg/Kg) consistent with its higher potency demonstrated in vitro. The beneficial effect of DOS was also monitored by clinical score, including behaviour, fur, breath, eyes and body weight.

Autoptic analysis revealed that lymphocytes infiltrated in tissues (e.g. lung) of DOS-treated mice showed high level of IFN gamma and IL10, i.e. cytokines involved in the inflammation and immune protection from lung viral infection (FIG. 17). All together these data identify DOS as a new class of compounds that can be added to next generation vaccine formulations to restore immunity in the elderly.

However, just the single treatment of DOS alone in vivo is sufficient to boost immune protection against lethal viral influenza infection in old mice, both immediately (2 days) after DOS treatment and in a long term protocol (8 weeks) (FIG. 18A). The IgG-H1N1 specific antibody titres and their neutralization effect was also restored in old mice on the DOS only regimen similarly to mice that were treated with the vaccine along with DOS46L (FIG. 16 and FIG. 18). FIG. 19 shows protection from lung necrosis in DOS46L treated animals compared to untreated age matched infected old controls. Consequently, in fact, real-time qPCR on lungs of DOS treated animals demonstrated a much lower amount of H1N1 viral titres than old mice that were not injected with DOS. The long-term rejuvenating effect is also observed by permanent stem-like generation in animals treated with DOS46L that were examined up to 8 weeks which may be related to the immune prophylactic effects of DOS.

DOS regimens may not only be complementary, but also an alternative to vaccination in virtue of complete immune rejuvenation and restoration of antigen specific function, resulting in much broader immune protection from viruses, as shown, for COVID (FIG. 24), cytomegalovirus (CMV) and varicella zoster virus (VZV) (FIG. 25, as well as protection from bacteria as for tuberculosis BCG vaccine. As such, this demonstrates that DOS compounds can be used as prophylactic anti-viral and anti-microbial compounds in general, as well as vaccine boosters.

It is well-known to the skilled person that cancer can escape the immune system by modulating checkpoint inhibitors on T cells, such as PD-1 and TIM-3 (Robert, Nature Communication 2020). Additionally, it has been well-documented in the literature that Sestrins are an important mediator in different kinds of cancer and diseases. Phenotypic analysis further showed that DOS-treated T cells down-regulated the immune-checkpoint inhibitor receptors PD-1 and TIM3 (FIG. 21), which are exploited by cancer for immune evasion and are responsible for checkpoint inhibitor resistance therapy. Therefore, DOS-induced checkpoint inhibitor downregulation may stop cancer cells from evading immune-responses. A combination therapy between checkpoint inhibitors and DOS, as well as DOS treatment on its own, may therefore be beneficial. For example, DOS compounds may act alone to induce death of cancer cells, especially neuroblastoma, a paediatric disease with very poor prognosis, and breast cancer (FIG. 22).

### CONCLUSIONS

The inventors have generated novel inhibitors (cyclic polypeptides) of sMAC, named DOS (disruptors of sMAC), and have demonstrated that these DOS compounds can inhibit sMAC in nanomolar amounts, by targeting sestrin for proteasomal degradation. Advantageously, the inventors demonstrated that the novel DOS compounds can provide long term immune protection against bacterial or viral infections with a single dose, both in the presence and absence of a vaccine. Furthermore, the inventors have shown that DOS compounds can reduce senescent T cells, and increase the levels of cytokines, such as IFN gamma, TNF alpha and IL-4 upon DOS treatment. Advantageously, therefore, this demonstrates the ability of DOS compounds to rejuvenate the aged immune system in the elderly, to prevent, treat or ameliorate a number of bacterial or viral infections, as well as age-related conditions, including cancer and neurodegenerative diseases.

1. A cyclic polypeptide, derivative or analogue thereof, comprising or consisting of an amino acid sequence derived from sestrin, or a truncation thereof.

2. The cyclic polypeptide, derivative or analogue thereof according to claim 1, wherein the cyclic polypeptide, derivative or analogue comprises or consists of an amino acid sequence derived from sestrin 1, or a truncation thereof, optionally wherein sestrin 1 comprises an amino acid sequence substantially as set out in SEQ ID No: 1, 2 or 3, or a variant or fragment thereof.

3. The cyclic polypeptide, derivative or analogue thereof according to claim 1, wherein the cyclic polypeptide, derivative or analogue comprises or consists of an amino acid sequence derived from sestrin 2, or a truncation thereof, optionally wherein sestrin 2 comprises an amino acid sequence substantially as set out in SEQ ID No: 4, or a variant or fragment thereof.

4. The cyclic polypeptide, derivative or analogue thereof according to claim 1, wherein the cyclic polypeptide, derivative or analogue comprises or consists of an amino acid sequence derived from sestrin 3, or a truncation thereof, optionally wherein sestrin 3 comprises an amino acid sequence substantially as set out in SEQ ID No: 5, 6 or 7, or a variant or fragment thereof.

5. The cyclic polypeptide, derivative or analogue thereof according to claim 3, wherein the cyclic polypeptide, derivative or analogue thereof comprises or consists of an amino acid sequence derived from the first 160 amino acids forming sestrin 2, or a truncation thereof.

6. The cyclic polypeptide, derivative or analogue thereof according to any preceding claim, wherein the cyclic polypeptide, derivative or analogue thereof is configured to bind to domain A, and/or at the interface between domain A and C of sestrin 1, sestrin 2, and/or sestrin 3.

7. The cyclic polypeptide, derivative or analogue thereof according to any preceding claim, wherein the cyclic polypeptide, derivative or analogue thereof comprises or consists of between 2 and 15 amino acid residues, more preferably between 3 and 9 amino acid residues, more preferably between 3 and 8 amino acid residues, and even more preferably between 4 and 7 amino acids.

8. The cyclic polypeptide, derivative or analogue thereof according to any preceding claim, wherein the cyclic polypeptide, derivative or analogue thereof comprises or consists of 5 amino acid residues.

9. The cyclic polypeptide, derivative or analogue thereof according to any preceding claim, wherein the cyclic polypeptide, derivative or analogue thereof comprises or consists of an amino acid sequence substantially as set out in any one of:
(i) SEQ ID Nos: 8 to 84, or a functional variant or fragment thereof;
(ii) SEQ ID Nos: 58, 59, 63, 65, 86, 87, 92, 94 to 97, 107, 113, 114, 116 to 119, 121 to 124, 126, 130, 133, 148, 150, 154, 155, 159 to 162, or a functional variant or fragment thereof;
(iii) SEQ ID Nos: 170 to 172, 174, 179, 187, 190, 196 to 199, or 202, or a functional variant or fragment thereof;
(iv) SEQ ID Nos: 58 to 60, 62, 64, 88 to 91, 93, 98 to 112, 115, 120, 125, 127 to 129, 131, 132, or 134 to 147, or a functional variant or fragment thereof;
(v) SEQ ID Nos: 47, 58 or 77, 62 to 65, 71 to 73, 149, 151 to 153, 156 to 158, 163 to 169, 173, 175, 176, 180 to 186, 188, 189, 191 to 195, 200, 201, or 203 to 205, or a functional variant or fragment thereof;
(vi) SEQ ID Nos: 89 to 91, 97, 206 to 218, or a functional variant or fragment thereof;
(vii) SEQ ID Nos: 176, 180, 219 to 234, or a functional variant or fragment thereof;
(viii) SEQ ID Nos: 235 to 247, or a functional variant or fragment thereof;
(viv) SEQ ID Nos: 248 to 260, or a functional variant or fragment thereof;
(vv) SEQ ID Nos: 58, 59, 62 to 65, or a functional variant or fragment thereof; or
(vvi) SEQ ID Nos: 261-269, or a functional variant or fragment thereof.

10. The cyclic polypeptide, derivative or analogue thereof according to any preceding claim, wherein the cyclic polypeptide, derivative or analogue thereof comprises or consists of an amino acid sequence substantially as set out in SEQ ID No: 22, 47 or 77, or a functional variant or fragment thereof.

11. The cyclic polypeptide, derivative or analogue thereof according to any preceding claim, wherein the cyclic polypeptide, derivative or analogue thereof consists solely of L-amino acids, or solely of D-amino acids, or both D- and L-amino acids.

12. A cyclic polypeptide, derivative or analogue thereof, comprising or consisting of an amino acid sequence selected from any of SEQ ID Nos: 8 to 84.

13. The cyclic polypeptide, derivative or analogue thereof according to any preceding claim, wherein the cyclic polypeptide, derivative or analogue thereof binds to and/or inhibits sestrin 1, sestrin 2 and sestrin 3, preferably domain A thereof.

14. The cyclic polypeptide, derivative or analogue thereof according to any preceding claim, wherein the cyclic polypeptide, derivative or analogue thereof:
(i) selectively binds to and/or inhibits sestrin 2;
(ii) comprises conserved amino acid residues between sestrin 1 and sestrin 2 upon blast search in the same position;
(iii) comprises conserved amino acid residues between sestrin 3 and sestrin 2 upon blast search in the same position;
(iv) comprises no conserved amino acid residues between sestrin 1 and sestrin 2 upon blast search in the same position;
(v) comprises no conserved amino acid residues between sestrin 3 and sestrin 2 upon blast search in the same position;
(vi) comprises a sestrin 1 region of homology (chemical similarities) in a different position;
(vii) comprises a sestrin 3 region of homology (chemical similarities) in a different position;
(viii) comprises a sestrin 1 sequence overlap and/or chemical similarities with sestrin 2;
(viv) comprises a sestrin 3 sequence overlap and/or chemical similarities with sestrin 2;
(vv) consists of or comprises sequences that completely overlap between all three sestrins; and/or
(vvi) consists of or comprises sequences that completely overlap between sestrin 1 and sestrin 3.

15. The cyclic polypeptide, derivative or analogue thereof according to any preceding claim, wherein the cyclic polypeptide, derivative or analogue thereof comprises or consists of formula (I)

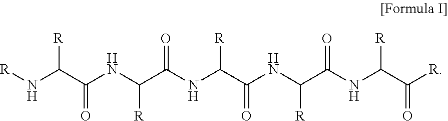

16. The cyclic polypeptide, derivative or analogue thereof according to claim 15, wherein the cyclic polypeptide, derivative or analogue thereof comprises binding or conjugation of:
(i) the terminal NH₂ and COOH groups (i.e. peptidic binding);
(ii) the NH₂ and NH₂ groups (i.e. hydrazine binding);
(iii) the NH₂ and CONH₂ groups (i.e. primary amidic binding);
(iv) the N═NH and NH₂ groups (i.e. hydrazine binding);
(v) the NH₂ and OH groups (i.e. hydroxylamine binding);
(vi) the NH₂ and SH groups (i.e. thiol-hydroxylamine binding);
(vii) the NH₂ and SR groups (i.e. hydrosulphide binding);
(viii) the COOH and CONH₂ groups (i.e. amide binding);
(ix) the COOH and COOH groups (i.e. dicarboxyl acid binding);
(x) the COOH and N═NH groups (i.e. carboxyl-amidogen binding);
(xi) the COOH and OH groups (i.e. hydroxy carboxylic binding);
(xii) the COOH and SH groups (i.e. thiol binding);
(xiii) the COOH and SR groups (i.e. thiol binding);
(xiv) the N═NH and CONH₂ groups (i.e. amidogen binding);
(xv) the N═NH and OH groups (i.e. N-hydroxylamine binding);
(xvi) the CONH₂ and CONH₂ groups (i.e. diamide binding);
(xvii) the CONH₂ and SR groups (i.e. s-amide binding);
(xviii) the SR and OH groups (i.e. hydroxy-thiol binding);
(xix) the SR and NH groups (i.e. thiol amine binding);
(xx) the NH₂ and NH groups (i.e. hydrazine binding);
(xxi) the COOH and NH groups (i.e. amino-propanoic binding);
(xxii) the SH and OH groups (i.e. hydrophilic binding);
(xxiii) the SH and NH groups (i.e. amino-thiol binding);
(xxiv) the OH and NH groups (i.e. hydroxylamine binding);
(xxv) the OH and OH groups (i.e. hydroxy binding);
(xxvi) the CONH₂ and OH groups (i.e. hydroxyamide binding);
(xxvii) the SH and SH groups (i.e. dithiol binding);
(xxviii) the CONH₂ and NH groups (i.e. hydroxyamide binding); or
(xxix) the NH and NH groups (i.e. diammonium binding),
to create the cyclic polypeptide, derivative or analogue thereof.

17. The cyclic polypeptide, derivative or analogue thereof according to any preceding claim, wherein the cyclic polypeptide, derivative or analogue thereof comprises or consists of a consensus or recurrentem sequence provided herein as SEQ ID No: 85, as follows:
[SEQ ID No: 85]
X₁X₂X₃X₄X₅
in which x₁ is A, P, H, L, R, M, D, F, V, I, E, N, Y, S, Q or T, preferably wherein x₁ is A, P, H, L, R, M or D;
x₂ is Y, A, L, R, M, F, K, V, E, I, D, S, Q, N or T, preferably wherein x₂ is Y, A, L or R;
x₃ is L, A, S, R, M, D, E, K, F, T, Q, C, V, P or N, preferably wherein x₃ is L, A, S or R;
x₄ is L, A, E, S, K, D, V, I, F, Q, H, C, T, P, N, R or Y, preferably wherein x₄ is L, A or E; and/or
x₅ is L, R, A, E, S, K, I, P, D, F, H, Y, C, T, N, Q, V or M, preferably wherein x₅ is L, R, A or E.

18. The cyclic polypeptide, derivative or analogue thereof according to any preceding claim, wherein cyclisation of the polypeptide, derivative or analogue thereof occur at amino acid residues 4, 9, 10, 15, 16, 19, 20, 21, 24, 26, 27, 28, 29, 30, 31, 32, 33, 34, 35, 36, 37, 39, 40, 41, 46, 50, 56, 61, 63, 65, 66, 67, 71, 73, 84, 91, 94, 96, 99, 103, 108, 109, 110, 131, 134, 147, 185, 204, 217, 220, 226, 228, 230, 233, 234, 236, 237, 238, 239, 240, 241, 244, 249, 251, 252, 255, 256, 272, 273, 274, 277, 284, 292, 298, 299, 301, 303, 304, 306, 309, 310, 311, 313, 314, 319, 331, 333, 334, 343, 359, 369, 431, 442, or 467 of sestrin 2 (SEQ ID No: 4), and/or amino acid residues 1-68, 72, 77, 78, 79, 84, 85, 88, 89, 90, 93, 95, 96, 97, 98, 99, 100, 102, 103, 104, 109, 113, 119, 124, 126, 128, 129, 130, 131, 135, 137, 148, 155, 158, 160, 163, 167, 172, 173, 174, 195, 198, 211, 249, 268, 281, 284, 290, 292, 294, 297, 298, 300 to 312, 315, 319, 320, 321, 323, 325, 326, 329, 330, 346, 347, 350, 357, 365, 371, 372, 374, 376, 378, 381, 382, 383, 385, 386, 391, 403, 405, 414, 430, 440, 502, 513, or 538 of sestrin 1 (SEQ ID No: 1).

19. The cyclic polypeptide, derivative or analogue thereof according to any preceding claim, wherein cyclisation of the polypeptide, derivative or analogue thereof occur at amino acid resides 1, 2, 4, 5, 10, 11, 14 to 29, 34, 35, 36, 39, 40, 41, 50, 56, 59, 61, 63, 66, 69, 70, 71, 81, 87, 91, 94, 96, 99, 103, 108, 109, 110, 131, 134, 142, 143, 147, 151, 180, 192, 204, 217, 220, 222, 224, 226, 228, 230, 231, 233, 234, 239, 240, 241, 243, 244, 245, 246, 249, 251, 255, 256, 272, 273, 277, 289, 299, 301, 302, 303, 306, 307, 310, 313, 314, 320, 331, 333, 334, 357, 359, 369, 380, 342, or 367 of sestrin 2 (SEQ ID No: 4), and/or amino acid residues 1 to 14, 16, 17, 22, 23, 30, 31, 32, 35, 36, 37, 46, 52, 55, 57, 59, 62, 65, 66, 67, 77, 83, 87, 90, 92, 95, 99, 104, 105, 106, 127, 130, 138, 138, 143, 147, 176, 188, 200, 213, 216, 218, 220, 222, 224, 226, 227, 229, 230, 232, 234, 235, 236, 237, 238, 239, 243, 244, 245, 247, 248, 249, 250, 253, 255, 259, 260, 276, 280, 292, 302, 304, 305, 308, 309, 312, 313, 314, 315, 316, 317, 318, 319, 320, 321, 322, 325, 326, 332, 343, 345, 346, 369, 371, 381, 392, 454, or 479 of sestrin 3 (SEQ ID No: 5).

20. The cyclic polypeptide, derivative or analogue thereof according to any preceding claim, wherein the cyclic polypeptide, derivative or analogue thereof targets sestrin in immune cells, preferably sestrin in B cells, Natural Killer (NK) cells, and/or T cells (T lymphocytes).

21. The cyclic polypeptide, derivative or analogue thereof according to claim 20, wherein the cyclic polypeptide, derivative or analogue thereof targets sestrin in CD4+ and/or CD8+ T cells, preferably sestrin in CD4+ CD27+ CD28+ cells and/or CD4+ CD27− CD28+ cells, and more preferably sestrin in CD4+ CD27− CD28− T cells (senescent T cells).

22. The cyclic polypeptide, derivative or analogue thereof according to any preceding claim, wherein the cyclic polypeptide, derivative or analogue thereof induces or restores cytokine production, preferably wherein the cyclic polypeptide, derivative or analogue thereof induces or restores the production of IFN gamma, TNF alpha and/or IL-4.

23. The cyclic polypeptide, derivative or analogue thereof according to any preceding claim, wherein the cyclic polypeptide, derivative or analogue thereof prevents or reverses immune-senescence.

24. The cyclic polypeptide, derivative or analogue thereof according to any preceding claim, wherein the cyclic polypeptide, derivative or analogue thereof is configured to rejuvenate the immune system.

25. The cyclic polypeptide, derivative or analogue thereof according to claim 24, wherein rejuvenating the immune system comprises: (i) restoring immune cell responsiveness to antigen stimulation; (ii) restoring telomere length and/or telomerase activity in immune cells; (iii) restoring antigen-specific proliferation and/or cytokine production in immune cells; and/or (iv) restoring antigen-specific antibody production by B cells and/or CD8+ T cells and/or Natural Killer cell expansion.

26. The cyclic polypeptide, derivative or analogue thereof according to any preceding claim, for use in therapy.

27. A nucleic acid encoding the cyclic polypeptide, derivative or analogue thereof according to any one of claims 1 to 25, for use in therapy.

28. The cyclic polypeptide, derivative or analogue thereof according to any one of claims 1 to 25, or a nucleic acid encoding the cyclic polypeptide, derivative or analogue thereof, for use in treating, preventing or ameliorating a microbial infection, an age-related condition, or a T-cell mediated disease.

29. The cyclic polypeptide, derivative or analogue thereof for use according to claim 28, wherein the microbial infection is bacterial, viral, fungal or protist.

30. The cyclic polypeptide, derivative or analogue thereof for use according to claim 28, wherein the age-related condition is a condition associated with, caused by, or related to, the aging and/or decline of the immune system, optionally wherein the age-related condition is characterised by sestrin-dependent sMAC activation and/or immune-senescence.

31. The cyclic polypeptide, derivative or analogue thereof for use according to claim 28, wherein the age-related condition is selected from a group consisting of cancer, such as paediatric neuroblastoma, melanoma, liver, pancreatic, testis or pancreatic cancer, smoke-induced emphysema, epilepsy, depression, metabolic diseases (including obesity), osteoporosis, erectile dysfunction, sterility, or neurodegenerative diseases, such as Alzheimer's disease or dementia.

32. A composition comprising the cyclic polypeptide, derivative or analogue thereof according to any one of claims 1 to 25, or a nucleic acid encoding the cyclic polypeptide, derivative or analogue thereof, and a vaccine.

33. The cyclic polypeptide, derivative or analogue thereof according to any one of claims 1 to 25, or a nucleic acid encoding the cyclic polypeptide, derivative or analogue thereof, for use in stimulating an immune response, preferably as a vaccine booster.

34. A pharmaceutical composition comprising a therapeutically effective amount of the cyclic polypeptide, derivative or analogue thereof according to any one of claims 1 to 25, or a nucleic acid encoding the cyclic polypeptide, derivative or analogue thereof, and optionally a pharmaceutically acceptable vehicle.

35. A process for making the pharmaceutical composition according to claim 34, the process comprising combining a therapeutically effective amount of the cyclic polypeptide, derivative or analogue thereof according to any one of claims 1 to 25, or a nucleic acid encoding the cyclic polypeptide, derivative or analogue thereof, with a pharmaceutically acceptable vehicle.
